SEQUENCE LISTING (1 sequence)

SEQ ID NO: 1 - protein, 8 aa, Artificial Sequence
  Synthetic albumin antigen
SIINFEKL

(19) United States
(12) Patent Application Publication
(10) Pub. No.: US 2011/0014292 A1
(43) Pub. Date: Jan. 20, 2011

(54) METHOD OF PROPHYLAXIS AND AGENTS FOR USE THEREIN

(75) Inventors: Robyn Elizabeth O'Hehir, Parkville (AU); Magda Plebanski, Clifton Hill (AU); Jennifer Rolland, Melbourne (AU); Charles Linton Hardy, Carlton North (AU)

Correspondence Address: KNOBBE MARTENS OLSON & BEAR LLP, 2040 MAIN STREET, FOURTEENTH FLOOR, IRVINE, CA, 92614, US

(73) Assignee: COOPERATIVE RESEARCH CENTRE FOR ASTHMA, Camperdown (AU)

(21) Appl. No.: 12/682,548

(22) PCT Filed: Oct. 15, 2008
(86) PCT No.: PCT/AU2008/001528

§ 371 Date: Sep. 27, 2010

(30) Foreign Application Priority Data
Oct. 15, 2007 (AU) 2007905630

(51) Int. Cl. A61K 9/14; A61K 31/785; A61P 11/06

(52) U.S. Cl. 424/489; 424/78.27

(57) ABSTRACT

The present invention relates generally to a method of prophylactically or therapeutically treating antigen-induced airway tissue inflammation and agents for use therein. More particularly, the present invention provides a method of prophylactically or therapeutically treating allergic airway inflammation and agents for use therein via the administration of the method of the present invention is useful, inter alia, in the treatment and/or prophylaxis of conditions characterised by antigen-induced airway tissue inflammation.

### FIELD OF THE INVENTION

The present invention relates generally to a method of prophylactically or therapeutically treating antigen-induced airway tissue inflammation and agents for use therein. More particularly, the present invention provides a method of prophylactically or therapeutically treating allergic airway inflammation and agents for use therein via the administration of the method of the present invention is useful, inter alia, in the treatment and/or prophylaxis of conditions characterised by antigen-induced airway tissue inflammation.

### BACKGROUND OF THE INVENTION

Bibliographic details of the publications referred to by author in this specification are collected alphabetically at the end of the description.

Inflammation of the lung tissue, despite being a normal part of the immune response, is nevertheless a potentially serious condition which, where very severe or even mild but chronic, can lead to significant and sometimes irreversible damage to the lung tissue. Still further, the onset of inflammation can be localised to one lung or it may spread to both.

The lung inflammatory process is characterised by inflammatory changes in large and small airways leading to damage of the alveoli and capillaries. In chronic inflammation, the repair of the epithelium is impaired resulting in mucus hypersecretion, airway narrowing and fibrosis and destruction of the parenchyma. The intensity and cellular characteristics of chronic airway inflammation varies as the disease progresses. Once inflammatory cells are activated, they release mediators which damage lung structures. These include a wide range of potent proteases (Shapiro, 1998), oxidants, and toxic peptides. Activation may further lead to the release of chemotactic peptides that perpetuate inflammation and tissue damage (Senior and Griffin, 1980, J Clin Invest, 66).

More specifically, the airways and lung parenchyma are found to exhibit high numbers of macrophages, T-lymphocytes (predominantly CD8+T cells), and neutrophils. Leukotriene B4 (LTB4), interleukin 8 (IL-8), and tumor necrosis factor-α (TNF-α) are the major inflammatory mediators involved in this process. The induction of this inflammatory response ultimately leads to:

### (i) Mucus Hypersecretion

(ii) Vascular damage
(iii) Airway narrowing or fibrosis; and
(iv) Elastin destruction.

In contrast to other types of injury and repair processes, the inflammation and tissue remodelling (fibrosis) observed in airway inflammation is often irreversible and may therefore exist through the life of the afflicted individual.

Pulmonary inflammation can be caused by a wide variety of factors including:
genetic predisposition
airway hyperresponsiveness
occupational dusts
indoor and outdoor air pollution
infections
autoimmunity.

Although inflammation is a normal and necessary part of an effective immune response, it can nevertheless quickly become very damaging if left unchecked or if induced in response to an innocuous antigen. The disease conditions which are characteristically associated with pulmonary inflammation include asthma, chronic obstructive pulmonary disease, cystic fibrosis, lung fibrosis, acute lung injury and ARDS.

In terms of airway hyperresponsiveness, despite increasing evidence that airborne particulate matter detrimentally affects lung function, much remains to be determined about the exact component(s) of particulate matter which are responsible, with roles attributed to particulates (fine and ultrafine), ozone and nitrogen dioxide (Brunekreef et al. 2002. Lancet 360:1233-1242; Heinrich et al. 2004, Curr Opin Allergy Clin Immunol 4:341-348; Gauderman et al. 2007, Lancet 369:571-577.). The particle concentration in ambient air is dominated by ultrafine particles (<100 nm in diameter) (Brunekreef et al. 2002, supra; Oberdorster, G. 2001, Int Arch Occup Environ Health 74:1-8). Ultrafine particles are postulated to contribute disproportionately to the morbidity and mortality associated with particle inhalation (Oberdorster 2001, supra; Bernstein et al. 2004, J Allergy Clin Immunol 114:1116-1123). Interest in the health effects of nanoparticles in the lung is also being driven by their increasing use in industrial and pharmaceutical applications. The evidence suggests that ultrafine particulate matter may play a disproportionately large role in asthma exacerbations (Peters et al. 1997, Am J Respir Crit Care Med 155:1376-1383; von Klot et al. 2002, Eur Respir J 20:691-702), and that nanoparticles of a variety of compositions instilled into the lung induce inflammation/toxicity (Brown et al. 2001, Toxicol Appl Pharmacol 175:191-199; Kaewamatawong et al. 2005, Toxicol Pathol 33:743-749; Renwick et al. 2004, Occup Environ Med 61:442-447; de Haar et al. 2005, Toxicol Sci 87:409-418). It is now clearly recognized that dendritic cells play a critical role in establishment and maintenance of pulmonary immune responses (Lambrecht and Hammad, 2003, Nat Rev Immunol 3:994-1003; Vermaelen and Pauwels, 2005, Am J Respir Crit Care Med 172:530-551). However, knowledge of the potential effects of interaction between nanoparticles with pulmonary antigen presenting cells/dendritic cells is scarce. Accordingly, there is a need to better understand the immunological responses of airway tissue to ultrafine particles in order to facilitate the development of effective therapeutic and prophylactic treatment regimes.

Particles are readily taken up by peripheral dendritic cells in vitro and in vivo, with ultrafine beads 40-100 nm showing preferential uptake (Fifis et al. 2004, J Immunol 173:3148-3154; Foged et al. 2005, Int J Pharm 298:315-322). Recent findings show that peripheral dendritic cells are sensitive to particles in the 40-50 nm size range, and when conjugated to antigen these nanobeads induce potent immune responses in small (Fifes et al. 2004, supra) and large animals (Scheerlinck et al. 2006, Vaccine 24:1124-1131). In addition, human lung-derived alveolar macrophages avidly internalize such nanobeads in vitro (Pouniotis et al. 2004, Clin Exp Immunol 143:363-372), thereby facilitating the onset of an immunological response. In work leading up to the present invention, however, it has been determined that in contrast to expectations based on published literature, inert nanobead instillation profoundly inhibits development of key parameters of antigen-induced airway inflammation. These findings present a new model for preventing or treating airway inflammation due to induction of a lung state resistant to the normal inflammatory triggers encountered upon antigen sensitization and challenge.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect there is provided a method of therapy or prophylaxis of antigen-induced airway tissue inflammation in a mammal said method comprising contacting said airway tissue with an effective amount of an ultrafine particle wherein said ultrafine particle induces or maintains non-inflammatory airway tissue homeostasis.

In another aspect there is provided a method of therapy or prophylaxis of antigen-induced lung tissue inflammation in a mammal said method comprising contacting said lung tissue with an effective amount of an ultrafine particle wherein said ultrafine particle induces or maintains non-inflammatory airway tissue homeostasis.

In yet another aspect there is provided a method of therapy or prophylaxis of allergen induced airway tissue inflammation in a mammal said method comprising contacting said airway tissue with an effective amount of an ultrafine particle wherein said ultrafine particle induces or maintains non-inflammatory airway tissue homeostasis.

In still another aspect there is provided a method of therapy or prophylaxis of antigen-induced airway tissue inflammation in a mammal said method comprising contacting said airway tissue with an effective amount of an inert 35 nm-55 nm particle.

In a further aspect there is provided a method of therapy or prophylaxis of antigen-induced airway tissue inflammation in a mammal said method comprising contacting said airway tissue with an effective amount of an inert particle of 45 nm, 46 nm, 47 nm, 48 nm, 49 nm, 50 nm, 51 nm, 52 nm, 53 nm, 54 nm or 55 nm.

Yet another aspect is directed to the use of an ultrafine particle in the manufacture of a medicament for the treatment or prophylaxis of antigen-induced airway tissue inflammation in a mammal wherein said ultrafine particle induces or maintains non-inflammatory airway tissue homeostasis.

### BRIEF DESCRIPTION OF THE DRAWINGS

FIG. 1 is an image depicting the effect of bead instillation on allergic airway inflammation. (A) Mice received beads or saline intratracheally twice, followed by sensitization with OVA/alum. Mice were challenged with saline or OVA on 4 occasions. (B) Differential analysis of absolute cell numbers in BAL. (C) Frequency of mucus-producing cells in airways as determined by PAS staining. (D) OVA-specific serum IgE. (E) ELISPOT analysis of Th2 cytokine production in lung-draining LN. Mean±SEM, 5-10 mice per group, data representative from 3-4 separate experiments. *p<0.05 no bead/OVA/OVA versus bead/OVA/OVA.

FIG. 2 is a graphical representation of the effect of nanobeads on CD11c and MHCII expression in trachea, lung and draining LN. Mice received FITC-labelled 47 nm beads or saline intratracheally prior to isolation of leukocytes from trachea, lung and draining LN at d1, d3 and d7 post-bead instillation. Following gating on forward and side scatter, gates were set on CD11c⁺MHCII⁺ and CD11c⁺MHCII^{hi} populations in (A) trachea, (B) lung and (C) draining LN. (D) Kinetic analysis of absolute numbers of indicated populations in trachea, lung and draining LN. Values calculated from mean absolute cell counts and percentage of each population as shown in A-C. n=9 mice per group per time-point, representative data from 2 separate experiments.

FIG. 3 is a graphical representation depicting nanobead uptake and CD205 expression on CD11c⁺MHCII^{+/hi} populations in the lung and draining LN. Mice received FITC-labelled 47 nm beads or saline intratracheally prior to isolation of leukocytes from lung and draining LN at d1, d3 and d7 post-bead instillation. Following gating on forward and side scatter, cells were gated on CD11c versus MHCII populations as defined in FIG. 2. Bead uptake (FITC⁺ events) and CD205 expression by (A) lung, and (B) draining LN leukocytes. n=9 mice per group per time-point, representative data from 2 separate experiments.

FIG. 4 is a graphical representation depicting the effect of nanobead treatment on co-stimulatory molecule expression by CD11c⁺CD11b^{hi} and CD11c⁺CD11b^{negative} populations in the lung. Mice received FITC-labelled 47 nm beads or saline intratracheally prior to isolation of lung leukocytes at d1, d3 and d7 post-bead instillation. (A) Following gating on forward and side scatter, gates were set for CD11c⁺CD11b^{hi} and CD11c⁺CD11b^{negative} populations. (B) Expression of CD40, CD80 and CD86 by the CD11c⁺CD11b^{hi} subset; filled histograms saline treated, empty histograms nanobead treated. n=9 mice per group per time-point, representative data from 2 separate experiments.

FIG. 5 is a graphical representation depicting nanobead uptake and CD11c expression by MHCII⁺F4/80^{negative} lung leukocytes. Mice received FITC-labelled 47 nm beads or saline intratracheally prior to isolation of lung leukocytes at d1, d3 and d7 post-bead instillation. (A) Following gating on forward and side scatter, gates were set on the MHCII⁺F4/80^{negative} population. (B) Bead uptake (FITC⁺ events) and CD11c expression by MHCII⁺F4/80^{negative} cells at d3 post-instillation. n=9 mice per group per time-point, representative data from 2 separate experiments.

FIG. 6 is a graphical representation depicting the effect of bead treatment and allergic airway inflammation on allergen uptake by tracheal leukocytes. Mice received 47 nm beads intratracheally prior to allergen sensitization and challenge with FITC-labelled allergen. Control mice received saline instead of beads or were saline sensitized. (A) Following gating on forward and side scatter, gates were set on FITC⁺CD11c⁺ cells. (B) Frequency of FITC⁺CD11c⁺ cells, and (C) expression of CD11b, MHCII and CD205 by FITC⁺CD11c⁺ cells. Pools of n=8-9 mice per group, representative data from 3 experiments with similar results.

FIG. 7 is a graphical representation depicting the effect of bead treatment and allergic airway inflammation on allergen uptake by lung parenchymal leukocytes. Mice received 47 nm beads intratracheally prior to allergen sensitization and challenge with FITC-labelled allergen. Control mice received saline instead of beads or were saline sensitized. (A) Following gating on forward and side scatter, CD11c⁺FITC⁺ events were gated as shown. (B) Expression of CD11b, MHCII and CD205 by FITC⁺CD11c⁺ cells. Pools of n=4-9 mice per group, representative data from 3 experiments with similar results.

FIG. 8 is a graphical representation depicting the effect of bead treatment and allergic airway inflammation on BAL fluid TGF-β concentrations and Foxp3 expression in lung and draining LN. Mice received 47 nm beads intratracheally prior to allergen sensitization and challenge with FITC-labelled allergen. Control mice received saline instead of beads or were saline sensitized. (A) BAL fluid TGF-β concentrations. Mean±SEM, n=7-10 mice per group. (B & C) Expression of Foxp3 by CD4⁺CD25⁺ cells from draining LN and lung. Mean±SEM, lung represents duplicate pools of 4-5 mice/group, draining LN represents triplicate pools of 3 mice/group. *p<0.05, saline/OVA/OVA versus beads/OVA/OVA, † p<0.01 saline/saline/OVA versus bead/saline/OVA, ¶ p<0.02 saline/saline/OVA versus saline/OVA/OVA.

FIG. 9 is a graphical representation depicting the effect of nanobead treatment on co-stimulatory molecule expression by CD11c⁺CD11b^{hi} and CD11c⁺CD11b^{negative} populations in draining LN. Mice received FITC-labelled 47 nm beads or saline intratracheally prior to isolation of LN leukocytes at d1, d3 and d7 post-bead instillation. (A) Following gating on forward and side scatter, gates were set for CD11c⁺CD11b^{hi} and CD11c⁺CD11b^{negative} populations. (B) Expression of CD40, CD80 and CD86 by the CD11c⁺CD11b^{hi} and CD11c⁺CD11b^{negative} subsets; filled histograms saline treated, empty histograms nanobead treated. n=9 mice per group per time-point, representative data from 2 separate experiments.

FIG. 10 is an image depicting the effect of nanobead treatment and allergic airway inflammation on pulmonary tissue cell counts and serum OVA-specific IgE. (A) Mice received either beads or saline intratracheally twice, followed by sensitization with saline/alum or OVA/alum. All mice were challenged with OVA×3 and OVA-FITC for the 4^{th} challenge. (B) Mean cell count per mouse from trachea (pool of 9 mice), lung (duplicate pools of 4-5 mice/group), draining LN (triplicate pools of 3 mice/group) and BAL (mean±SEM for 8-9 mice). (C) OVA-specific serum IgE. Mean±SEM, n=7-10 mice per group. *p<0.05, saline/OVA/OVA versus bead/OVA/OVA. Data representative of 3 separate experiments.

FIG. 11 is a graphical representation depicting the effect of bead treatment and allergic airway inflammation on allergen uptake by draining LN leukocytes. Mice received 47 nm beads intratracheally prior to allergen sensitization and challenge with FITC-labelled allergen. Control mice received saline instead of beads or were saline sensitized. (A) Following gating on forward and side scatter, CD11c⁺FITC⁺ events were gated as shown. (B) Expression of CD11b, MHCII and CD205 by FITC⁺CD11c⁺ cells. Pools of n=3-9 mice per group, representative data from 3 experiments with similar results.

FIG. 12 is a schematic diagram depicting the switching between complex lung states characterized by different APC subset distribution using total CD11c⁺DC and the CD11c⁺CD11b⁺ myeloid subset as examples. Changes in other CD11c⁺ subsets, particularly MHCII, F4/80 and CD205 expression are detailed in the text. Changes in numbers of a given cell population are illustrative of relative percentages. TOP: Intratracheal nanobead instillation alters proportions of total CD11c⁺ cells in trachea, lung or LN, and increased the proportion of CD11c⁺DC that co-express CD11b⁺ (mainly in the lung). BOTTOM: Effect of nanobead treatment or allergen sensitization on uptake of fluorescently labelled allergen by pulmonary APC. In non-sensitized animals bead treatment induces a moderate increase in the proportion of allergen⁺CD11c⁺DC that co-express CD11b. Upon systemic sensitization, there is a dramatic loss of allergen⁺CD11c⁺ DC from the lung with a pronounced increase in trachea and to a lesser degree in LN. Most of these cells co-express CD11b⁺. Lungs in these animals are inflamed (pink shading) characterized by airway eosinophilia and mucus secretion. By contrast, compared to control mice, nanobead pretreated animals only show moderate increases in allergen⁺CD11c⁺ cells that co-express CD11b⁺ across all compartments. Inflammation in these animals is greatly attenuated. Analysis of Foxp3⁺ Treg shows that nanobead treated animals retain Treg in the lung and draining LN, whereas these cells are greatly diminished in mice which did not receive beads prior to allergen challenge. Treg were only analyzed in the allergen challenged animals.

FIG. 13 depicts the e of nanoparticles and microparticles on allergic airway inflammation. (A) Mice received saline, 50 nm or 500 nm particles (labelled as nano or micro, respectively) prior to OVA sensitisation and challenge. (B) Differential analysis of absolute cell numbers in BAL, (C) total lung leukocytes, and (D) OVA-specific serum IgE ELISA. (E) Frequency of IL-13 producing lung-draining LN cells stimulated with medium or OVA. n=10 mice/group, representative of two separate experiments.

FIG. 14 is a graphical representation of the effect of nanoparticles on Th2 cytokine production, airway inflammation and mucus secretion. Mice received nanoparticles or saline i.t. prior to OVA sensitisation and saline or OVA challenge. (A) Frequency of IL-4, IL-5 and IL-13 producing cells lung draining LN cells stimulated with medium or OVA. n=2-5/cytokine (pooled from 6-10 mice/group). (B) Differential analysis of absolute cell numbers in BAL and (C) frequency of mucus-producing cells in airways as determined by PAS staining. n=6-10 mice/group.

FIG. 15 is a graphical representation of the effect of nanoparticles in mice with and without allergic airway inflammation. Mice received nanoparticles or saline i.t. prior to saline or OVA sensitisation and OVA challenge. (A) Differential analysis of absolute cell numbers in BAL, (B) total lung leukocytes, and (C) OVA-specific serum IgE. n=5-10 mice/group, representative of 3-4 separate experiments. (D) Frequency of IL-4, IL-5 and IL-13 producing lung-draining LN cells stimulated with medium or OVA. n=7-12 per cytokine (pooled data from 3 separate experiments).

FIG. 16 is a graphical representation of nanoparticle effects in C57BL/6 mice. Mice received nanoparticles or saline i.t. prior to OVA sensitisation and challenge. (A) Differential analysis of absolute cell numbers in BAL. n=7 mice/group. (B & C) Frequency of IL-4 and IL-5 producing lung-draining LN cells stimulated with medium or OVA. n=3-4 per cytokine (pooled from 7 mice/group).

FIG. 17 is a graphical representation of nanoparticle effects with the clinically-relevant Bermuda grass allergen or with peptide-coated nanoparticles. Mice received nanoparticles or saline i.t. prior to BGP sensitisation/challenge. (A) Differential analysis of absolute cell numbers in BAL. n=7 mice/group. (B) Frequency of IL-5 producing lung draining LN cells stimulated with medium or BGP. n=5 per cytokine (pooled from 7 mice/group). (C) Mice received nanoparticles-conjugated to SIINFEKL peptide or saline i.t. prior to OVA sensitisation/challenge. Frequency of IL-4 producing lung-draining LN cells stimulated with medium or OVA. n=7 mice/group.

FIG. 18 is an image depicting the effect of nanoparticles and allergic airway inflammation on MHCII and CD11b expression in the lung. Mice received nanoparticles or saline i.t. prior to saline or OVA sensitisation and challenge with FITC-labelled OVA. (A) Gating strategy for identification of viable OVA-FITC⁺CD11c⁺ cells. (B) Expression of CD11b and MHCII by OVA-FITC⁺CD11c⁺ cells. n=3 (each group consisting of pools of 4-9 mice), representative of 2 separate experiments.

FIG. 19 is a graphical representation of the effect of 50 nm particles and allergic airway inflammation on Treg frequencies and BAL fluid TGF-β. Mice received nanoparticles or saline i.t. prior to saline or OVA sensitisation and OVA challenge. (A) Frequency of CD4⁺CD25⁺Foxp3⁺ cells among viable draining LN cells, and (B) Foxp3 expression by CD4⁺CD25⁺ cells in lung and draining LN. n=2 or 3 for lung and LN respectively (pooled from n=9 mice/group). (C) BAL fluid TGF-β concentrations. n=7-10 mice/group.

### DETAILED DESCRIPTION OF THE INVENTION

The present invention is predicated, in part, on the determination that whereas ultrafine particles are known to induce airway inflammation, in particular allergic airway inflammation, a subgroup of such particles can in fact facilitate the induction or maintenance of normal airway tissue homeostasis. Accordingly, this finding has facilitated the development of methods of prophylactically or therapeutically treating conditions characterised by antigen induced airway inflammation, which conditions have, to date, often been treated with corticosteroids, in order to reduce inflammatory symptomology. This is generally recognised as a necessary but nevertheless undesirable treatment regime due to the side-effects associated with prolonged administration of corticosteroids. Accordingly, in addition to providing an effective means of achieving an immunological outcome which has not been attainable to date, the method of the present invention is also very simple to routinely perform and avoids the use of treatment regimes associated with unwanted side effects, such as corticosteroid treatment.

Accordingly, in one aspect there is provided a method of therapy or prophylaxis of antigen-induced airway tissue inflammation in a mammal said method comprising contacting said airway tissue with an effective amount of an ultrafine particle wherein said ultrafine particle induces or maintains non-inflammatory airway tissue homeostasis.

By reference to “airway tissue” is meant the tissue of the passages which run from the mouth and nose, including the mouth and nose, into the lungs, together with the alveoli. The largest of the passages which runs from the oral and nasal cavities is the trachea (also known as the “windpipe”). In the chest, the trachea divides into two smaller passages termed the bronchi, each of these being further characterised by three regions termed the primary bronchus, secondary bronchus and tertiary bronchus. Each bronchus enters one lung and divides further into narrower passages termed the bronchioles. The terminal bronchiole supplies the alveoli. This network of passages is often colloquially termed the “bronchial tree”. Without limiting the present invention in any way, the predominant cell types in the pseudostratified columnar tracheal and bronchial epithelia include basal, intermediate, goblet, and ciliated cells. The simple columnar epithelia of bronchioles contain two main cell types termed Clara and ciliated cells. The most distal and functionally specialised epithelia of the lung include the gas exchanging air spaces; squamous type I pneumocytes and cuboidal type II pneumocytes.

In one embodiment, said airway tissue is lung tissue.

According to this embodiment there is provided a method of therapy or prophylaxis of antigen-induced lung tissue inflammation in a mammal said method comprising contacting said lung tissue with an effective amount of an ultrafine particle wherein said ultrafine particle induces or maintains non-inflammatory airway tissue homeostasis.

Without limiting the present invention to any one theory or mode of action, the inflammatory response is a complex response characterised by a series of physiological and/or immunological events which are induced to occur by the release of a cytokine cascade in response to any one of a variety of stimuli including, but not limited to, tissue injury, infection, an immune response (such as to a pathogen or an innocuous agent—as occurs with allergies), or disease (such as tumour formation or an autoimmune response).

The physiological events which characterise inflammation include:

(i) vasodilation
(ii) increased vascular permeability
(iii) cellular infiltration
(iv) changes to the biosynthetic, metabolic and catabolic profiles of affected organs
(v) activation of the cells of the immune system.

It should therefore be understood that reference to an “inflammatory response” is a reference to any one or more of the physiological and/or immunological events or phases that are induced to occur in the context of inflammation and, specifically, in response to the signals generated by the cytokine cascade which directs the inflammatory response.

For example IL-1, TNFα and IL-6 are well known for their functions as pro-inflammatory mediators. It should also be understood that an inflammatory response within the context of the present invention essentially includes a reference to a partial response, such as a response which has only just commenced, or to any specific phase or event of a response (such as the phases and events detailed in points (i)-(v), above, or any other effect related to inflammation including, but not limited to, the production of acute phase proteins—including complement components and fever). Reference to a “chronic” inflammatory response should be understood as a reference to a response which is not acute. More specifically, it is of a prolonged duration, such as weeks, months or even indefinitely. An “acute” inflammatory response, however, is a reference to the immediate and early response to tissue injury such as physical, chemical or microbial insult. An acute inflammatory response is usually complete within a short duration, typically hours to a few days.

Without limiting the present invention to any one theory or mode of action, in certain circumstances the acute inflammatory process, characterized by neutrophil infiltration and oedema, gives way to a predominance of mononuclear phagocytes and lymphocytes. This is thought to occur to some degree with the normal healing process but becomes exaggerated and chronic when there is ineffective elimination of foreign materials as occurs in certain infections (e.g. tuberculosis) or following introduction of foreign bodies (e.g. cigarette smoke) or deposition of crystals (e.g. urate crystals). Chronic inflammation is often associated with fusion of mononuclear cells to form multinucleated gigant cells, which eventually become a granuloma. Chronic inflammation is also seen under conditions of delayed hypersensitivity.

In terms of the present invention, it has been determined that antigen induced airway tissue inflammation can be prophylactically or therapeutically treated by administering an ultrafine particle which can induce or maintain normal airway tissue homeostasis. By “antigen” is meant any proteinaceous or non-proteinaceous molecule which is capable of inducing an immune response in the airway tissue, this inherently involving the onset of airway tissue inflammation. Examples of such antigens include, but are not limited to, pathogens (such as viral, bacterial or parasitic), tobacco related particles, environmental particles, plant derived particles (such as pollens), chemical or other synthetic form of pollutant (such as airborne pollutants present in smog), other airborne particles (such as dust related allergens) or organism derived particles (such as house dust mite faeces). It should be appreciated that the subject allergen may be one which is generally expected to function as a foreign, immunogenic molecule, such as a chemical molecule found in pollutants, or it may be one which is innocuous, such as grass pollen. The nature of the immune response which is generated may take any form. For example, immune responsiveness to some innocuous allergens often takes the form of a delayed type hypersensitivity reaction while immune responsiveness to other classes antigens may take the form of a hypersensitivity response which is not delayed or it may take the form of another class of immune response which is not regarded as a typical hypersensitivity response, such as a cell mediated response to a virus.

Accordingly, in one embodiment there is provided a method of therapy or prophylaxis of allergen induced airway tissue inflammation in a mammal said method comprising contacting said airway tissue with an effective amount of an ultrafine particle wherein said ultrafine particle induces or maintains non-inflammatory airway tissue homeostasis.

In another embodiment there is provided a method of therapy or prophylaxis of pathogen induced airway tissue inflammation in a mammal said method comprising contacting said airway tissue with an effective amount of an ultrafine particle wherein said ultrafine particle induces or maintains non-inflammatory airway tissue homeostasis.

Examples of such pathogens includes, but is not limited to, respiratory syncytial virus, rhinovirus, influenza virus, cytomegalovirus and parainfluenza virus.

Without limiting the present invention to any one theory or mode of action, the administration of ultrafine particles in accordance with the method of the invention is characterised by extensive redistribution of dendritic cell subsets across lung compartments, particularly in the airways. This state is characterized by low effector T cell responses (both Th1 and Th2), but maintenance of normal (baseline) CD4⁺CD25⁺Foxp3⁺Treg frequencies during local allergen challenge. Accordingly, reference to “homeostasis” in accordance with the method of the present invention should be understood as a reference to the maintenance of an airway tissue physiological state which is a non-inflammatory state. To this end, it should be understood that said homeostasis is intended as a reference only to inflammatory-related homeostasis of the airway tissue and not to the homeostasis of other physiological factors, unrelated to inflammation, such as pulmonary related homeostasis, surfactant-related homeostasis and the like.

The ultrafine particles of the present invention are preferably inert. By “inert” is meant that the particles are substantially devoid of toxic contaminants.

As detailed hereinbefore, it has been determined that a subpopulation of ultrafine particles can induce or maintain non-inflammatory airway tissue homeostasis. An “ultrafine” particle should be understood as a particle of less than 100 nm. As exemplified herein, this subpopulation includes inert 30 nm-70 nm ultrafine particles. Still more preferably, said particles are from 35 nm-65 nm and yet more particularly 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm or 64 nm. Most particularly, said particles are 45 nm, 46 nm, 47 nm, 48 nm, 49 nm, 50 nm, 51 nm, 52 nm, 53 nm, 54 nm or 55 nm.

The present invention therefore more particularly provides a method of therapy or prophylaxis of antigen-induced airway tissue inflammation in a mammal said method comprising contacting said airway tissue with an effective amount of an inert 35 nm-55 nm particle.

Yet more particularly there is provided a method of therapy or prophylaxis of antigen-induced airway tissue inflammation in a mammal said method comprising contacting said airway tissue with an effective amount of an inert particle of 45 nm, 46 nm, 47 nm, 48 nm, 49 nm, 50 nm, 51 nm, 52 nm, 53 nm, 54 nm or 55 nm.

It should be understood that with respect to the subject ultrafine particle there is no particular limitation on the shape or surface morphology that the ultrafine particles may take. Generally, the particles will be spherical or spheroidal in shape. For avoidance of any doubt, reference to the “size” of the particles is intended to be that of the largest dimension provided by a cross section of a given particle. Thus, in the case of spherical particles the size is the diameter of the sphere, as measured to the outer perimeter of the sphere.

The particles may be in the form of primary particles, or in the form of an aggregation of primary particles. Generally, the particles will be in the form of primary particles.

The structure of the particle may be homogeneous or heterogeneous in terms of composition and also in terms of the physical state of the constituent components that form the composition. For example, the structure of the particles may be formed from one or more components that are in a solid state. The particles may also have a core-shell type structure in which the outer shell is formed from one or more components that are in a solid state and the inner core is formed from one or more components that are in a liquid state. Having said this, it will be appreciated that in order to function in accordance with the invention, the particles will at least have an outer surface or shell that is formed from one or more components that are in a solid state. Generally, the particles will be formed from one or more components that are in a solid state.

As used herein, reference to a component of the particles being in a “solid” or “liquid” state is meant that the component has that physical state at a temperature of no less than that which would be experienced by the particle when in vivo (i.e. generally at a temperature of no less than about 37° C.).

The particles may be formed from any suitable material provided that it does not promote a toxic response when used in accordance with the invention. In other words, at the very least the outer surface of the particles that makes contact with lung tissue will be formed from, or coated/grafted with, an inert material. Examples of suitable materials that the particles may be formed from or coated with include, but are not limited to, polymer, inorganic material such as ceramic and glass, metal or an organic material, such as glycine.

The particles are preferably made from a polymeric material. The polymeric material may or may not be biodegradable. In the context of the present invention, by a polymeric material being “biodegradable” is meant that the physical structure of the polymeric material is degraded in vivo such that the polymer can ultimately be excreted from the host. Degradation of the polymeric material may occur via physical or chemical pathway. Where a biodegradable polymer is to be used, its degradation products should not be toxic to the host. Examples of polymeric materials from which the particles may be formed include, but are not limited to, polystyrene, polyacrylates, polymethacrylates, polyolefins such as polypropylene and polyethylene, polyfluorocarbons such as Teflon, polyurethanes, polyamides, polycarbonates and polyesters. Suitable biodegradable polymers include, but are not limited to, biodegradable polyurethanes, biodegradable polyesters and biodegradable polycarbonates. The outer surface of the particles may be provided with functional groups that can be used to alter the surface characteristics of the particles. For example, the functional groups may be used to provide a charge at the particle surface or they may be used as a reaction site to tether or graft a surface modifying agent to the particle. Such functional groups may include, but are not limited to, amine groups, carboxyl groups, hydroxyl groups and sulfate groups. Examples of surface modifying agents that may be tethered or grafted at the surface of the particles include, for example, amino acids, such as glycine. Techniques for tethering or grafting surface modifying agents to the surface of a substrate such as a particle are generally well known in the art.

The particles may also comprise one or more therapeutic agents such as a pharmaceutically active compound. Upon being positioned within the lung, such particles may be designed to release the agent into the host in a controlled manner. For example, the agent might be dispersed throughout the polymeric matrix of a polymer particle and diffuse from polymer matrix in a desired manner into the lung.

Particles suitable for use in accordance with the invention may be prepared using known techniques. The particles may also be obtained commercially. For example, suitable particles may be purchased from Polysciences Inc. Warrington, Pa., USA under the Tradename Polybead®.

In yet another aspect there is provided a method of therapy or prophylaxis of a condition characterised by antigen-induced airway tissue inflammation in a mammal said method comprising contacting said airway tissue with an effective amount of an ultrafine particle wherein said ultrafine particle induces or maintains non-inflammatory airway tissue homeostasis.

Preferably, said airway tissue is lung tissue. Still more preferably, said antigen is an allergen. Yet more preferably said ultrafine particle is an inert particle of 30 nm-70 nm, more preferably 35 nm-65 nm and most preferably about 45 nm, 46 nm, 47 nm, 48 nm, 49 nm, 50 nm, 51 nm, 52 nm, 53 nm, 54 nm or 55 nm.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animals (eg. kangaroos, deer, foxes). Preferably, the mammal is a human or a laboratory test animal. Even more preferably, the mammal is a human.

The method of the present invention is useful as a therapeutic or a prophylactic treatment. By prophylactic treatment is envisaged the administration of said ultrafine particles in individuals who have not yet developed antigen-induced airway tissue inflammation but may, for example, be at risk of developing such a condition. In this regard, and without limiting the present invention in any way, it has been demonstrated that where said ultrafine particles are administered to non-inflamed airway tissue, this tissue is able to maintain its non-inflammatory homeostasis in the face of subsequent antigen challenge. In the context of a therapeutic treatment regime, the method of the present invention can reduce the level of inflammation, thereby inducing a shift back towards normal non-inflammatory homeostasis. Accordingly, it should be understood that reference to maintaining or reducing non-inflammatory airway tissue homeostasis is a reference to not just entirely preventing the onset of airway inflammation or eliminating pre-existing inflammation but also to at least partially reducing said inflammation or, in the context of the prophylactic aspects of this invention, reducing the extent or severity of the onset of an airway inflammatory state.

Accordingly, reference herein to “treatment” and “prophylaxis” is to be considered in its broadest context. The term “treatment” does not necessarily imply that a subject is treated until total recovery. This is a particularly significant point in relation to the present invention since in the context of disease conditions in which airway tissue inflammation is one of a range of symptoms, other symptoms may not be alleviated by this method. In this situation, the method of the invention is “treating” the disease condition in terms of reducing or eliminating the occurrence of a highly undesirable symptom but may not eliminate other symptoms unrelated to inflammation which may nevertheless be induced by the antigen. For example a toxic antigen, such as a pollutant, may nevertheless exert other systemic outcomes associated with the toxicity of the antigen itself. Similarly, “prophylaxis” does not necessarily mean that the subject will not develop some symptomology. However, the method of the present invention may slow or reduce the onset or degree of inflammation. The term “prophylaxis” may therefore be considered as reducing the severity or onset of a particular condition. “Treatment” may also reduce the severity of an existing condition. To this end, the nanoparticles of the present invention may therefore be administered as a pretreatment to the onset of the condition in issue. In this context, for example, the nanobeads may be administered prior to vaccination with the antigen (such as a pathogen or allergen) after vaccination with the antigen or subsequently to infection.

Yet another aspect is directed to the use of an ultrafine particle in the manufacture of a medicament for the treatment or prophylaxis of antigen-induced airway tissue inflammation in a mammal wherein said ultrafine particle induces or maintains non-inflammatory airway tissue homeostasis.

Preferably, said airway tissue is lung tissue. Still more preferably, said antigen is an allergen. Yet more preferably said ultrafine particle is an inert particle of 30 nm-60 nm, more preferably 35 nm-65 nm and most preferably about 45 nm, 46 nm, 47 nm, 48 nm, 49 nm, 50 nm, 51 nm, 52 nm, 53 nm, 54 nm or 55 nm.

There are a wide range of well characterised conditions which are associated with antigen induced airway inflammation. For example, allergens induce airway hypersensitivity, such as Type I hypersensitivity, and pathogens cause infection, such as viral infection, bacterial infection or parasitic infection. The inhalation of other types of particulate matter, such as the particles present in tobacco smoke, smog or other pollution can induce inflammation associated with one or more of a range of conditions such as asthma, emphysema, COPD, acute respiratory distress syndrome, pneumonia, acute lung injury, lung fibrosis and bronchiectasis.

The present invention is preferably achieved by administering to said mammal an effective amount of a modulatory agent as hereinbefore defined. To this end, an “effective amount” means an amount necessary to at least partly attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

The present invention further contemplates a combination of therapies, such as the administration of the modulatory agent together with other proteinaceous or non-proteinaceous molecules which may facilitate the desired therapeutic or prophylactic outcome. For example, in the context of the therapeutic treatment of asthma, one may seek to maintain ongoing anti-inflammatory therapies until such time as the method of the present invention has become effective.

Administration of the ultrafine particles of the present invention hereinbefore described, in the form of a pharmaceutical composition, may be performed by any convenient means. The particles of the pharmaceutical composition is contemplated to exhibit therapeutic or prophylactic activity when administered in an amount which depends on the particular case. The variation depends, for example, on the human or animal and the modulatory agent chosen. A broad range of doses may be applicable. Dosage regimens may be adjusted to provide the optimum response. For example, several divided doses may be administered daily, weekly, monthly or other suitable time intervals or the dose may be proportionally reduced as indicated by the exigencies of the situation.

The modulatory agent may be administered in any convenient or suitable manner although respiratory routes are preferred. For example, one may administer by inhalation or insufflation of powders or aerosols (including by nebulizer); intratracheal or intranasal.

For inhalation, the composition of the invention can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In accordance with these methods, the composition defined in accordance with the present invention may be coadministered with one or more other compounds or molecules. By “coadministered” is meant simultaneous administration in the same formulation or in two different formulations via the same or different routes or sequential administration by the same or different routes. For example, the subject particles may be coadministered together with anti-inflammatory or other relevant drugs in the context of asthma treatment. By “sequential” administration is meant a time difference of from seconds, minutes, hours or days between the administration of the two types of molecules, These molecules may be administered in any order.

Yet another aspect of the present invention is directed to the use of an ultrafine particle for the therapeutic or prophylactic treatment of antigen-induced airway tissue inflammation in a mammal.

The present invention is further described by reference to the following non-limiting examples.

### Example 1

### Materials and Methods

### Mice

Female BALB/c mice aged 7-8 weeks were obtained from Laboratory Animal Services (Adelaide, South Australia) and housed in the Alfred Medical Research and Education Precinct animal facility. Numbers of mice per group are indicated in the Figure legends. All experimental protocols were approved by the precinct Animal Ethics Committee.

### Bead Preparation and Immunizations

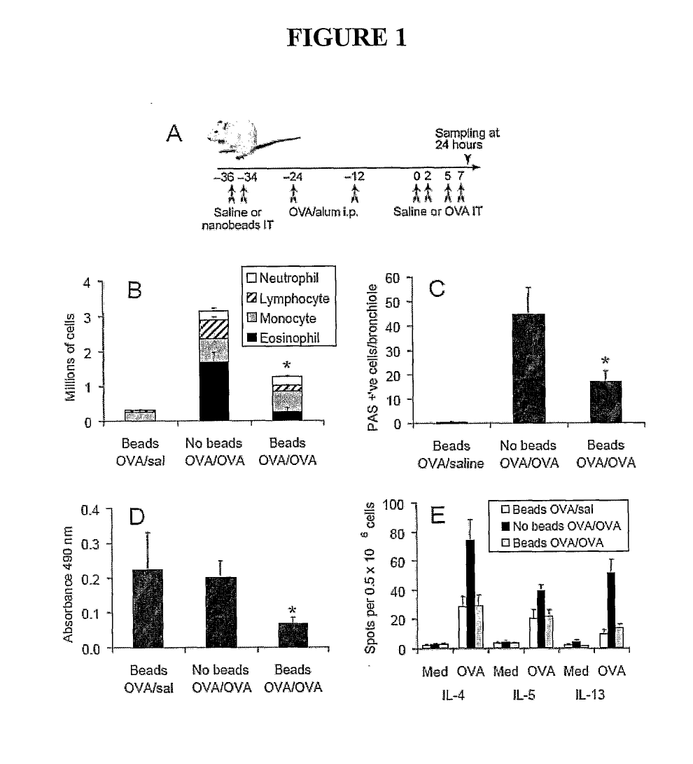

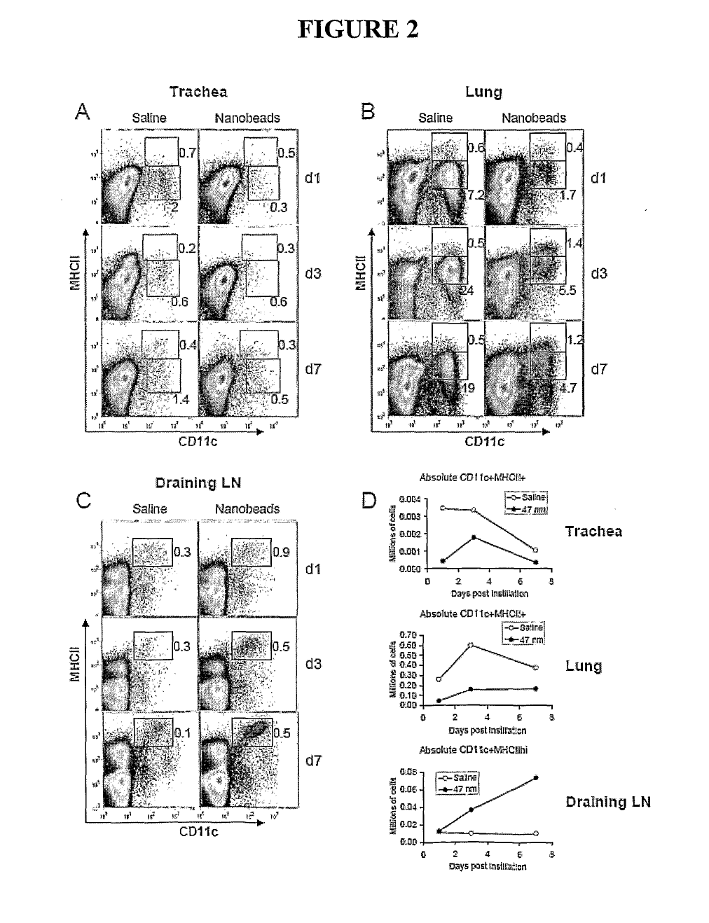

Mock bead conjugation was performed as follows. Polybead carboxylate microspheres (0.0471 μm; Polysciences Inc. Warrington, Pa. USA #15913) were added to a glass tube at 1% solids (@ 1.46×10¹⁴ particles/ml) and sonicated for 5 minutes. MES buffer (2-[N-Morpholino]ethanesulfonic acid; MP Biomedicals Irvine, Calif. USA #195309) was added to 50 mM and the pH adjusted to 6. EDAC (N-Ethyl-N′-(3-dimethylaminopropyl) carbodiimide hydrochloride; Sigma-Aldrich, Castle Hill NSW #E1769) was added to 4 mg/ml, and pH adjusted to 6.5. The beads were mixed at room temperature for 2 hours. Glycine (Sigma-Aldrich #G7126) was added to 7 mg/ml, and mixed for 30 minutes. The beads were dialysed overnight against PBS at 4° C., and sonicated in a water bath sonicator prior to use. In certain experiments mice received FITC-labelled particles (20 μg in 50 μl saline) or saline as control; pilot experiments indicated that the effects of unlabeled versus FITC-labelled beads were indistinguishable (data not shown). Beads prepared under GLP conditions with endotoxin below the level of detectability of the LAL assay similarly gave indistinguishable results from beads prepared under normal laboratory conditions (data not shown). On days −36 and −34 mice were anaesthetised and nanobeads (20 μg) or saline delivered intratracheally (50 μl). On days −24 and −12 mice were sensitized intraperitoneally with saline or OVA (50 μg; Sigma-Aldrich) adsorbed to aluminium hydroxide. Mice were challenged intratracheally with saline or OVA (25 μg) on days 0, 2, 5 and 7 as described previously (Hardy et al. 2003, Am J Respir Crit Care Med 167:1393-1399). In some experiments 25 μg FITC-conjugated OVA (Molecular Probes, Eugene, Oreg., USA #023020) was used for the final (4^{th}) challenge. Mice were killed 24 hours after the final challenge (FIGS. 1A and 2A). Challenge with OVA or OVA-FITC elicited identical pulmonary allergic inflammatory responses.

### Tissue Sampling and Cell Isolation

Methods were as described previously (Hardy et al. 2003, supra). Blood was collected from the inferior vena-cava and serum collected. LN suspensions were prepared by gently grinding through a 70 μm cell strainer (BD Falcon, San Jose, Calif., USA #352350). BAL was performed with 0.4 ml 1% FCS in PBS and 3 further lavages of 0.3 ml. Viable LN and BAL cells were counted in a hemocytometer. For differentials, BAL cytospots were Giemsa-stained (Merck, Kilsyth Victoria) and ≧200 cells identified by morphological criteria.

For flow cytometric analysis we used an enzymatic tissue digestion protocol as described (Vremec et al. 1992, J Exp Med 176:47-58) with modifications. The right ventricle was perfused with 5 ml Ca²⁺/Mg²⁺-free HBSS (Invitrogen, Mt. Waverly Victoria #14175095) with 0.01 M EDTA, pH 7.2. The trachea including major bronchi as they entered the lung were dissected free from the lung, and draining LN, trachea and lung tissue separately minced with a scalpel blade on ice. Tissue fragments were pelleted by centrifugation (10 minutes, 350 g at 4° C.). LN, trachea and lung were digested in collagenase type III (1 mg/ml; Worthington, Lakewood, N.J. USA) and DNase type I (0.025 mg/ml; Roche Diagnostics, Sydney NSW #1284932) at 25° C. in the dark mixing continuously; after 1 hour fresh collagenase/DNase solution was added and digestion continued for 1 hour. The reaction was stopped by adding one 10^{th} volume of EDTA and 3% FCS and mixing for 5 minutes. The cell suspension mixed with a pipette to break up clumps and filtered through a 70 μm cell strainer (BD Falcon), red cells lysed, and washed twice in staining buffer [3% FCS, 3% pooled normal mouse serum, 5 mM EDTA (pH 7.2) and 0.1% Na-Azide in Ca²⁺/Mg²⁺-free HBSS]. Viable tracheal, lung, and LN leukocytes were counted in a hemocytometer.

### Flow Cytometry

Non-specific FcR binding was blocked by incubating cells in CD16/CD32 block (BD Biosciences, San Jose, Calif., USA) and 3% pooled normal mouse serum in EDTA-containing staining buffer (see above). Cells were stained on ice for 20 minutes with combinations of the following antibodies/conjugates diluted in staining buffer (all BD unless noted): CD11b-PE, CD11c-APC, CD40-biotin, CD80-biotin, CD86-biotin, class II MHC (I-A^{d})-biotin, DEC-205-PE (Cedarlane, Hornby, Ontario, Canada), F4/80-PE (Caltag, Burlingame, Calif. USA), and streptavidin-PerCP. Appropriate isotype control antibodies were used. Cells were protected from light at all times. Approximately 1×10⁶ events were acquired on a FACSCalibur™ (BD), and analyzed on FlowJo (Tree Star, Ashland, Oreg. USA).

### Intracellular Foxp3 Staining

One million cells/well were plated in a 96 well V-bottom plate and blocked with FACS staining buffer for 30 min on ice. Surface staining was performed with CD4-PerCP and CD25-PE or isotype control (IgG2_{b}-PE) diluted in staining buffer for 30 min on ice, followed by two washes in staining buffer. Cells were incubated in 100 μl/well fixation/permeablisation buffer (eBiosciences #88-8118) for 30 min on ice and washed once with permeablisation buffer. Cells were intracellularly stained with Foxp3-APC antibody or isotype control (rat IgG2ₐ-APC) diluted in permeablisation buffer+2 μl normal mouse serum for 30 min on ice, washed once and resuspended in 1% paraformaldehyde.

### Cytokine ELISPOT

IL-4, IL-5, and IL-13 ELISPOT were performed as described previously (Hardy et al. 2003, Clin Exp Allergy 36:941-950). IFNγ ELISPOT was performed using AN18 capture and R4-6A2 biotinylated detection antibodies (Mabtech, Mossman, NSW Australia; #3321-3-1000 and #3321-6-1000) and hydrophobic membrane plates (Millipore #MAIPS4510).

### OVA-Specific IgE ELISA

OVA-specific IgE was detected as described previously (Hardy et al. 2003, supra). Briefly, ELISA plates were coated with OVA (10 μg/ml) and incubated with IgG-depleted serum diluted 1:5, followed by anti-mouse IgE-biotin and streptavidin-peroxidase. Absorbance was read at 490 nm; results are expressed as raw OD readings minus background (no serum added).

### Cytokine ELISA

BAL fluid was acid activated prior to detection of TGF-β according to the manufacturer's instructions (R&D Systems #DY1679). The limit of detection was 8 pg/ml. For IL-10 analysis, IL-10 capture and detection antibodies (#551215 and #554465, respectively, BD) were used according to the manufacturer's instructions. BAL fluid was used neat, 1:2 and 1:4. Detection was performed with streptavidin-HRP (Amersham Biosciences, #RPN1231) and reaction product developed with 3,3′,5,5;-TetraMethylBenzidine (Zymed, Calif. USA, #00-2023). The reaction was stopped with an equal volume of 1M HCl and plates read at 450 nm.

### Quantitation of Airway Mucus Production

Formalin-fixed paraffin embedded lung sections were stained with periodic acid-Schiff (PAS) reagent. The numbers of PAS-positive cells per small bronchiole (approx. basement membrane circumference 0.5 mm) were counted. Six to ten airways were counted per mouse.

### Statistical Analysis

Statistics were analysed using SPSS 12.0.1 software. The Mann-Whitney U test or paired Student's t-Test were used as appropriate, with differences considered statistically significant at p<0.05.

### Results

### Nanobead Instillation Inhibits Allergic Pulmonary Inflammation

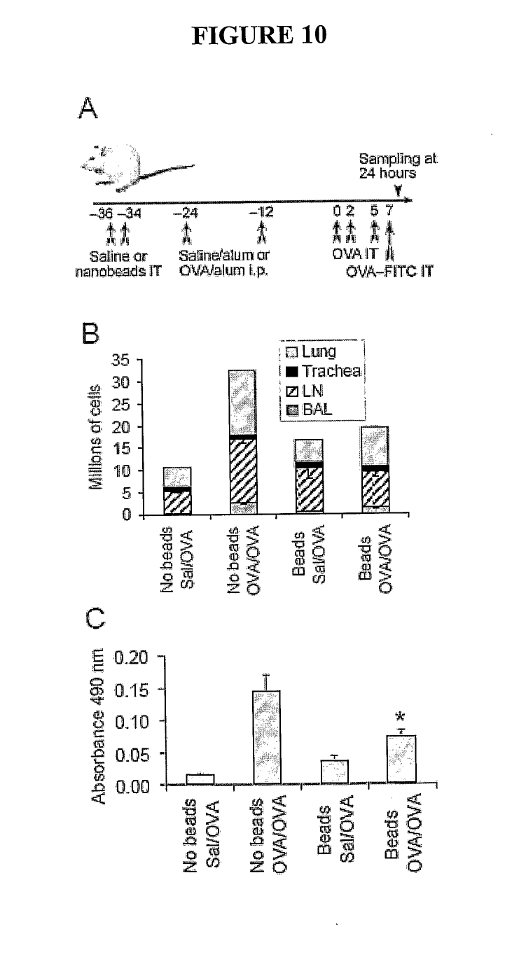

To test the hypothesis that ultrafine particle instillation in the lung promotes susceptibility to allergic lung inflammation 47 nm nanobeads were instilled intratracheally into the mouse lung 12 days prior to sensitization with OVA/alum, and mice were subsequently (36 days later) challenged intratracheally with OVA, or saline as control (FIG. 1A). As expected in this model of allergic inflammation, mice which received saline prior to OVA sensitization and challenge (saline/OVA/OVA) had a 10-fold increase in the number of bronchoalveolar lavage (BAL) cells, comprised mainly of eosinophils, and a 110-fold increased frequency of mucus-producing cells in the airways (FIGS. 1B & 1C). In marked contrast and contrary to expectations, mice that received nanobeads prior to OVA sensitization (beads/OVA/OVA) not only failed to have more severe inflammatory responses than the saline/OVA/OVA group, but had in fact 2.5-fold fewer total BAL cells, a>6-fold decreased eosinophil count (FIG. 1B), and approximately 3-fold decreased airway mucus-producing cell frequency and OVA-specific IgE concentrations (FIGS. 1C & 1D). Generally, beads/OVA/saline mice resembled naïve animals with airway leukocytes consisting predominantly of macrophages with scant eosinophils (0.1%) and negligible mucus-producing cells; these animals did, however, produce OVA-specific IgE, due to the OVA sensitization (FIG. 1D) whereas saline/alum-sensitized mice had very low serum IgE levels (FIG. 10C). These findings demonstrate that inert nanobeads instilled into the airways prior to OVA sensitization/challenge prevent the induction of cardinal features of allergic airway inflammation. Our data also show that nanobeads instilled into the airways of allergen sensitized mice, in the absence of allergen challenge, do not induce pulmonary inflammation or goblet cell hyperplasia.

### Nanobead Instillation Inhibits Th2 Cytokine Production in the Draining LN

Antigen-specific Th2 cells play a critical role in development of allergic airway inflammation. We asked whether the induction of local Th2 immunity (in lung draining LN) would be affected by nanobead instillation. The frequency of OVA-stimulated draining LN cells producing the Th2 cytokines IL-4, IL-5 and IL-13 in the saline/OVA/OVA group were all at ≧78 cells per 10⁶ cells (FIG. 1E). In marked contrast, beads/OVA/OVA mice had a 2- to 4-fold decreased frequency of LN cells producing IL-4, IL-5 and IL-13, with values similar to control beads/OVA/saline (unchallenged ‘healthy’ mice). The decreased production of these cytokines is further consistent with the observed decreased OVA-specific IgE, airway eosinophilia and goblet cell frequency, respectively. Immune deviation, by the induction of robust antigen-specific Th1 response, has been shown to be capable of inhibiting Th2-biased immune responses.

The frequency of cells producing IFN-γ in both the beads/OVA/OVA and saline/OVA/OVA groups was comparable and low at <20 per 10⁶ cells (data not shown). These results show that nanobeads instilled into the airways of allergen sensitized mice impair the induction of a Th2-biased response in the draining LN and that this is not as a consequence of immune deviation and associated increased IFN-γ production.

### Nanobead Instillation Alters Distribution of APC in Distinct Pulmonary Compartments

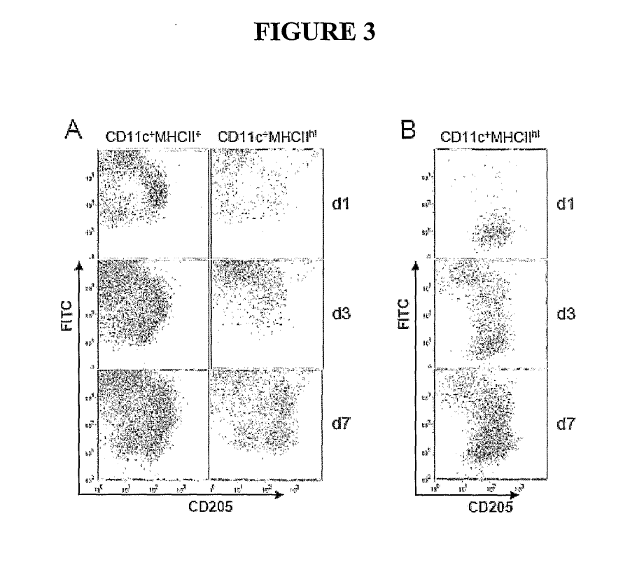

An alternative potential mechanism by which nanobeads could prevent the induction of airway inflammation by decreasing Th2 immunity would be by a direct effect on APC in the lung, particularly DC. To address this possibility we instilled naïve mice with FITC-labelled nanobeads to determine their short and long-term effects on lung APC and included for the analysis a range of general APC and DC subset markers. We used saline instillation as control. Lung DC are not a homogeneous population with DC from trachea (representing airways) and parenchyma having distinct functional and phenotypic properties (Huh et al. 2003, J Exp Med 198:19-30; von Garnier et al. 2005, J Immunol 175:1609-1618). We therefore determined separately the consequences of nanobead instillation on leukocyte numbers and phenotype from trachea, lung parenchyma and draining LN. The main findings were that bead instillation caused a marked reduction in frequency of CD11c⁺MCHII⁺DC in tracheal digests particularly at d1 following instillation, and this effect persisted to d7 (FIGS. 2A and 2D). Similarly in the lung, nanobeads induced a loss of CD11c⁺MHCII^{hi} DC and CD11c⁺MHCII⁺ macrophages (FIGS. 2B and 2D). In striking contrast, CD11c⁺MHCII^{hi} myeloid DC (mDC) proportions and absolute numbers were increased 8-fold in the draining LN by d7 (FIGS. 2C and 2D). In the lung particles were taken up by both macrophage-like cells (CD11c⁺MCHII⁺) and mDC (CD11c⁺MHCII^{hi}) which had down-regulated the endocytic marker CD205, with bead-positive (FITC⁺) proportions peaking at d3 (FIG. 3A). Increased proportions of FITC⁺CD205^{negative} cells were also seen by CD11c⁺MHCII^{hi} mDC in the draining LN with the peak also at d3 (FIG. 3B).

Nanobead Instillation Increases Proportions of CD11b^{hi} DC

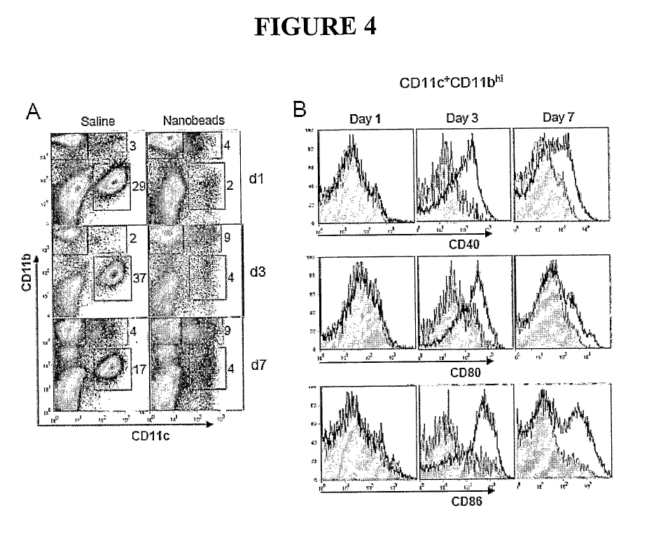

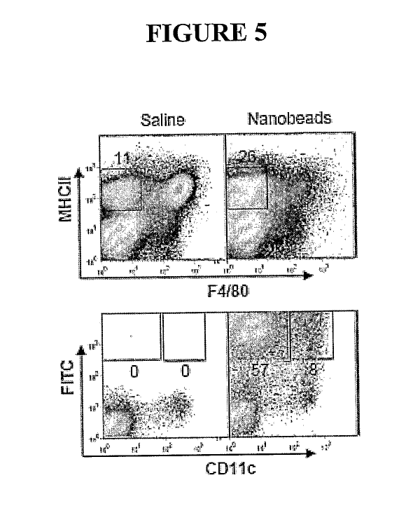

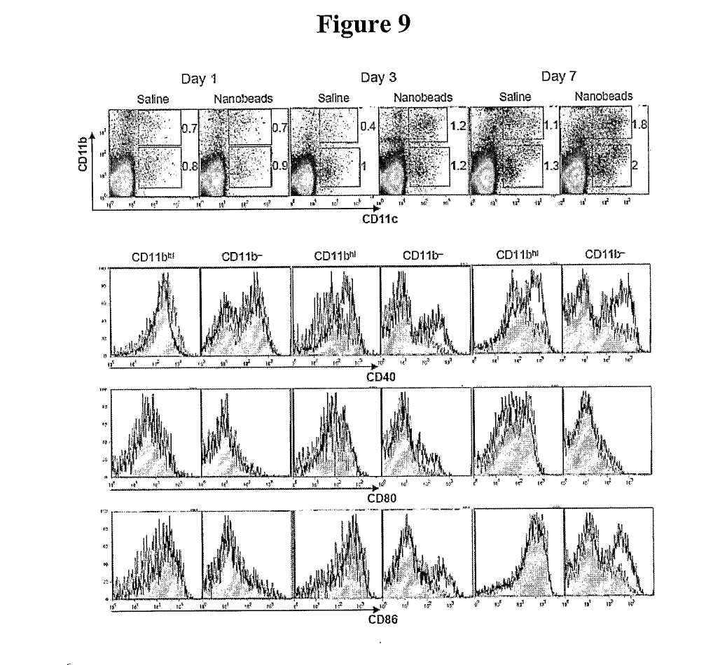

Consistent with others (von Garnier et al. 2005, supra; Wikstrom and Stumbles 2007, Immunol Cell Biol 85:182-188), we found a population of CD11c⁺ cells which express CD11b at high density. Particle instillation increased the proportion of lung CD11c⁺CD11b^{hi} cells from 2-3% of total cells in normal mice to 9% by d3 and d7 post-instillation (FIG. 4A), while a more subtle increase (<2-fold) occurred in the airways. In parallel, we noted a dramatic loss of CD11c⁺CD11b^{lo/negative} cells in the lung (FIG. 4A) and a simultaneous increased frequency of MHCII⁺F4/80^{negative} cells (from 6% to 17% at d1, 11% to 26% at d3, 11% to 15% at d7; saline versus particles, respectively), of which a significant proportion (74% at d1, 57% at d3 and 20% at d7) were FITC⁺CD11c^{negative} (FIG. 5). Nanobead instillation also increased proportions of CD11c⁺CD11b^{hi} cells in the draining LN approximately 2-fold at all time points, while there was no change in frequency of the CD11c⁺CD11b^{negative} subset (FIG. 9). The proportion of nanobead-laden CD11c⁺CD11b^{hi} cells peaked at d3 with up to 46% and 76% being FITC⁺ in the airways and lung, respectively, dropping to approximately 18% and 46% by d7. Similar kinetics were seen in the draining LN where FITC⁺CD11c⁺CD11b^{hi} cells increased from 6% at d1 to 41% at d3, and dropped to 22% at d7. The proportion of CD11c⁺CD11b^{negative} cells in the draining LN which took up nanobeads was generally 2-3-fold lower than the CD11b^{hi} subset.

### Nanobead Instillation Transiently Up-Regulates Co-Stimulatory Molecule Expression

Nanobead-induced changes in the composition of the DC ‘milieu’ across different pulmonary immune compartments may result in differential migration or maturational status. By d3 lung parenchymal CD11c⁺CD11b^{hi} cells had increased CD40, CD80 and CD86 mean fluorescence intensity 2-4 fold, and this decreased slightly by d7 (FIG. 4B). Distinct T cell co-stimulatory molecule expression profiles were seen for draining LN CD11c⁺CD11b^{hi} and CD11c⁺CD11b^{negative} populations, with CD40 mean fluorescence intensity increased 1.5- and 2-fold at d3 and d7, respectively, by the CD11c⁺CD11b^{hi} subset, and 2- to 3-fold increases in CD40 and CD86 expression by the CD11c⁺CD11b^{negative} subset at d7 (FIG. 9). Collectively these data show that nanobeads are taken up primarily by CD11c⁺CD11b^{hi} DC in the airway/lung inducing transient co-stimulatory molecule up-regulation; these cells accumulate in the draining LN with similar kinetics where they display increased CD40 and CD86.

### Nanobead Pretreatment Alters Subsequent Patterns of Allergen Uptake and APC Migration Across Lung Compartments

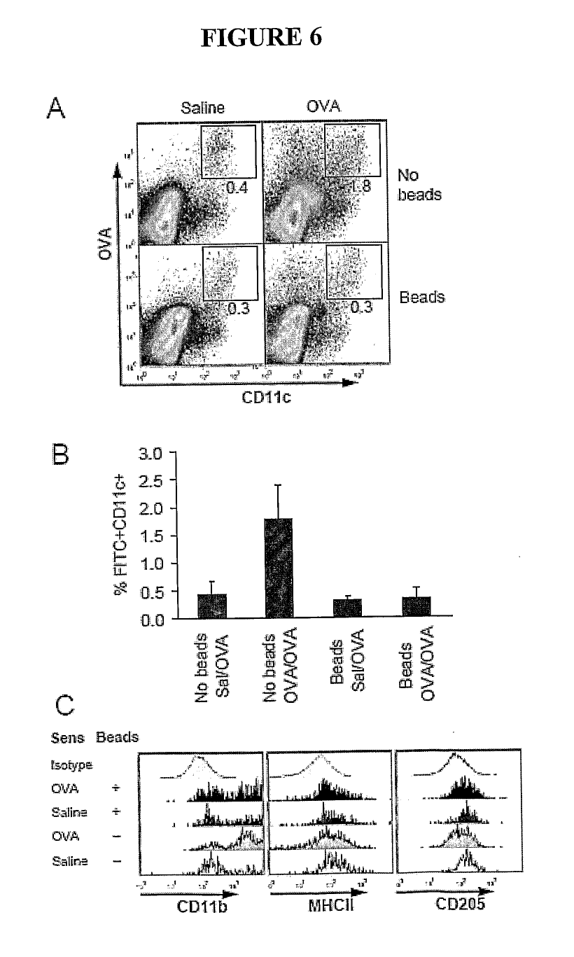

We hypothesized that the above noted re-distribution of APC across lung compartments in turn results in different and/or less APC taking up intratracheally instilled allergen explaining the downstream lack pulmonary Th2 immunity. To determine whether particles alter APC migration during acute allergic airway inflammation mice received particles prior to systemic OVA sensitization, and were challenged with OVA intratracheally and FITC-labelled OVA at the 4th challenge thereby permitting tracking of pulmonary APC which have endocytosed FITC-labelled allergen (Vermaelen et al. 2001, J Exp Med 193:51-60; Vermaelen et al. 2003, Am J Respir Cell Mol Biol 29:405-409) (FIG. 10A). As shown above, nanobead treatment decreased total BAL counts, but also decreased draining LN and lung cell counts (FIG. 10B). As seen above, serum OVA-specific IgE titres were reduced approximately 2-fold compared to saline pre-treatment (FIG. 10C). Having confirmed that this model reproduced our key findings we used it to explore the pattern of allergen uptake by lung APC. Leukocytes were separately isolated from airway (trachea), lung parenchyma and draining LN. Pilot experiments showed that the lung parenchymal FITC⁺CD11c⁺ population consisted almost entirely of cells with macrophage/DC morphology; the FITC⁺CD11c^{negative} population contained a mixture of cell types including small lymphocytes with numerous dying/apoptotic cells, and the FITC^{negative}CD11c⁺ population contained a large proportion of eosinophils (data not shown). Compared to mice which did not receive nanobeads or OVA sensitization (saline/saline/OVA) the frequency of FITC⁺CD11c⁺ cells in the sensitized and challenged group (saline/OVA/OVA) in the trachea was increased 4-fold (FIGS. 6A & 6B). Strikingly, nanobead instillation (beads/OVA/OVA) completely prevented this increase such that the FITC⁺CD11c⁺ cell frequency was identical to both the saline/saline/OVA and beads/saline/OVA groups. These data also show that nanobead instillation per se did not cause an increase in airway FITC⁺CD11c⁺ DC in the trachea. Similar results were obtained when we determined frequencies of CD11c⁺MHCII⁺ and CD11c⁺MHCII^{hi} cells. Airway inflammation increased the frequency of CD11b^{hi} FITC⁺CD11c⁺ cells from 17±7% to 63±11% (saline/saline/OVA versus saline/OVA/OVA, respectively, p<0.05), and this increase was substantially prevented by nanobead instillation (44±9%, beads/OVA/OVA, p<0.05). MHCII expression was uniformly low in all groups of mice, consistent with a relatively immature status of airway DC. The frequency CD205^{hi} FITC⁺CD11c⁺ cells was decreased by airway inflammation (70-75%, saline/saline/OVA and beads/saline/OVA versus 45% in the saline/OVA/OVA, p<0.05) and this was partially prevented by bead treatment (57%, bead/OVA/OVA, p<0.01).

Nanobead Instillation Prevents Increases in CD11b^{hi} and MHCII^{hi} Allergen-Laden Cells in the Lung but not the Draining LN

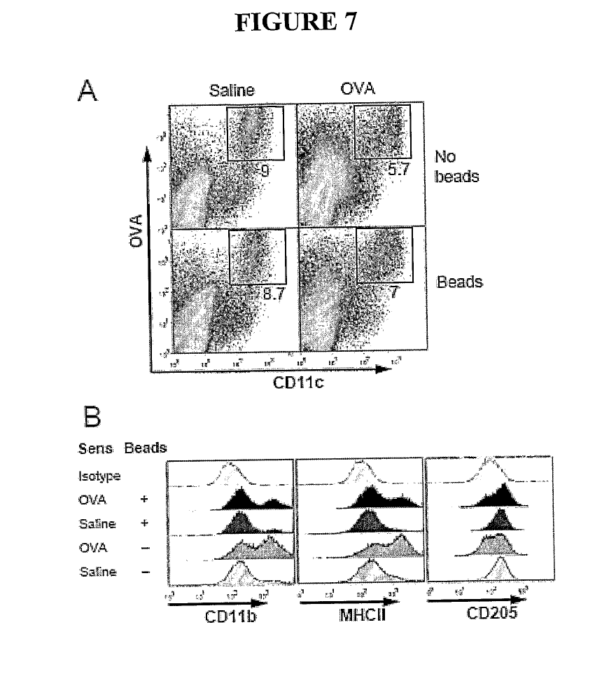

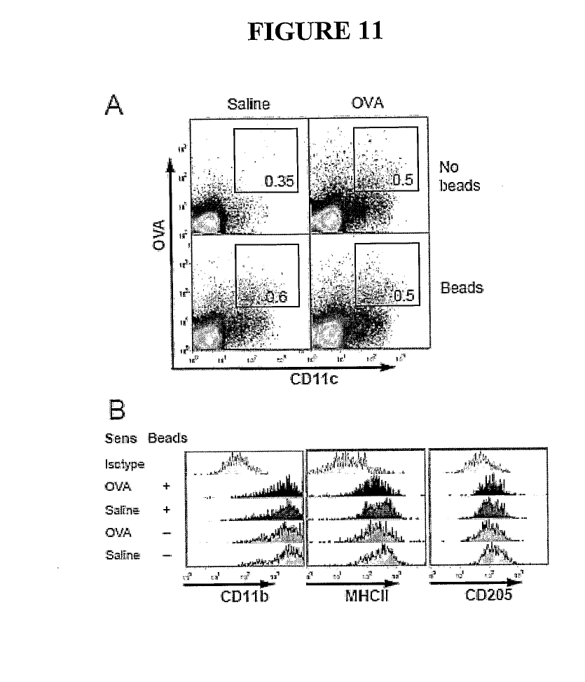

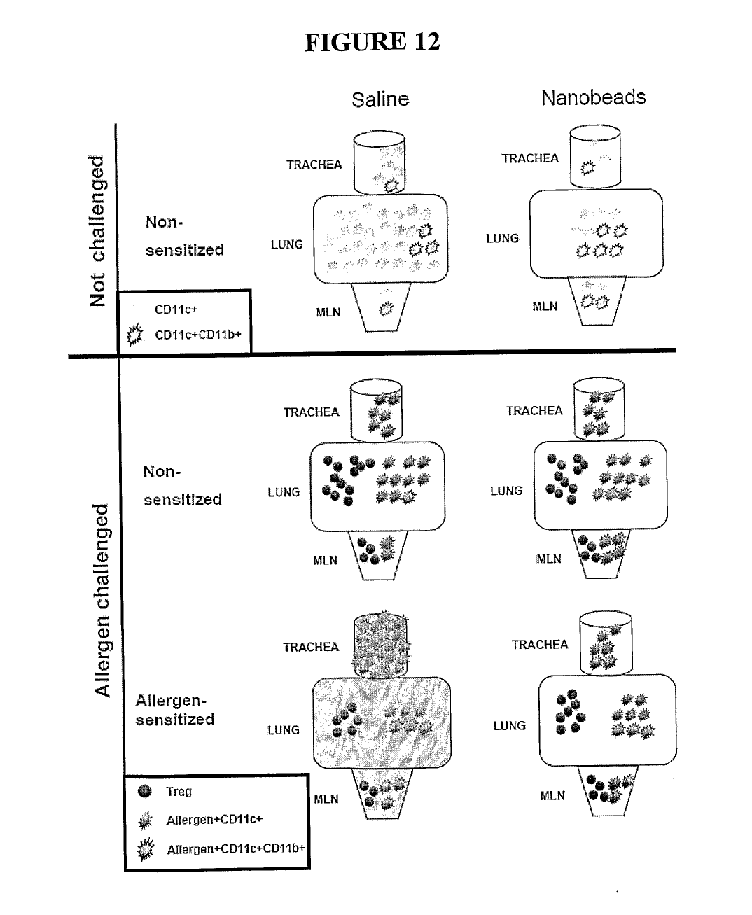

Dramatic differences between DC subpopulations in the airways could be an independent local effect, or may also be reflected in differential DC distribution in the lung and draining LN. The proportions of FITC⁺CD11c⁺ cells in the lung were decreased in the saline/OVA/OVA group (5.7±1%) compared to the control no inflammation group (9±1.5%, saline/saline/OVA, p<0.05), and nanobead instillation partially prevented this (7±1%, beads/OVA/OVA). The low frequency of CD11b^{hi} FITC⁺CD11c⁺ cells in the ‘no inflammation’ groups (10±2% and 17±4%, saline/saline/OVA and beads/saline/OVA, respectively) was markedly increased by airway inflammation (51±2%, saline/OVA/OVA, p<0.001) and this was partially prevented by nanobead instillation (35±5%, p<0.05, FIGS. 7A & 7B). Similarly, while FITC⁺CD11c⁺ cells in the no inflammation groups had a low frequency of MHCII^{hi} cells (18±4% and 11±3%, saline/saline/OVA and beads/saline/OVA, respectively), this was increased to 49±6% in mice with airway inflammation (saline/OVA/OVA) and this was partially prevented by nanobeads (31±7%, p<0.01, FIG. 7B). The % CD205^{hi} FITC⁺CD11c⁺ cells in the control groups was 61±1% and 62±2% (saline/saline/OVA and beads/saline/OVA, respectively), while this was decreased in the airway inflammation group (45±2%, saline/OVA/OVA, p<0.02) and this was prevented by bead treatment (60±7%, beads/OVA/OVA). In the draining LN nanobead instillation increased proportions of FITC⁺CD11c⁺ cells in the no inflammation group (0.35±0.1 versus 0.6±0.09, saline/saline/OVA versus bead/saline/OVA, respectively, p<0.05). However, FITC⁺CD11c⁺ cells were uniformly CD11b^{hi}, MHCII^{hi} and CD205⁺, irrespective of immunisation or nanobead treatment status (FIG. 11). These data show that nanobead instillation profoundly affected DC subset distribution and activation status, primarily in the airways and lung, but had little effect on phenotype in the draining LN.

### Nanobead Instillation and the Restoration of Foxp3-Positive Cell Frequency

Rapid redistribution of APC and specifically CD11c⁺ DC from lung and trachea to the LN results in a novel homeostatic condition resistant to induction of allergic airway inflammation, with dramatically reduced allergen uptake locally in the airway/trachea by CD11c⁺ DC. Limited effector T cell stimulation is observed downstream, in particular an absence of the expected Th2 immunity elicited locally even in the presence of peripheral sensitization (as evidenced by IgE induction in the bead/OVA/saline group in FIG. 1D).

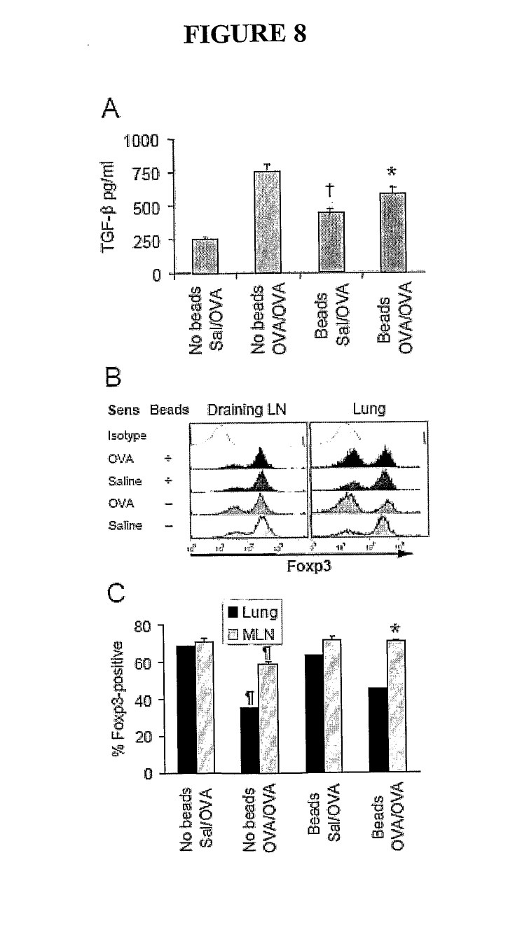

Although this novel mechanism explains all the findings, we used the same particle treatment and allergen sensitisation/challenge protocol to explore the possibility that bead treatment could promote regulatory cytokines and/or regulatory T cells (Treg) in the lung or lung-draining LN. Nanobead treatment induced modest decreases in BAL fluid concentrations of the immunomodulatory cytokine TGF-β(saline/OVA/OVA versus beads/OVA/OVA, p<0.05), while nanobeads instilled into mice without airway inflammation caused slightly increased TGF-β concentrations (p<0.01, FIG. 8A). No change in BAL fluid IL-10 concentrations was observed in any of the groups (data not shown). We examined intracellular Foxp3 expression on CD4⁺CD25⁺ cells and found uniformly high frequencies of positive cells in the draining LN in the negative control groups with no inflammation (71±2% and 72±1%, saline/saline/OVA and beads/saline/OVA, respectively), while this was clearly decreased by airway inflammation (58±2%, saline/OVA/OVA, p<0.02). Nanobeads completely prevented this loss of Treg such that they remained at control levels (71±1%, saline/OVA/OVA versus beads/OVA/OVA, p<0.05, FIGS. 8B & 8C). Similarly in lung parenchymal leukocytes, a high proportion of CD4⁺CD25⁺ cells were Foxp3⁺ in the groups with no inflammation with 69±2% and 63±3% for saline/saline/OVA and beads/saline/OVA, respectively. This was decreased 2-fold in the saline/OVA/OVA group (35±4%, p<0.02) and this decrease was partially prevented by nanobead instillation (45±3%, beads/OVA/OVA, FIGS. 8B & 8C). These results show that nanobead instillation supported maintenance of Foxp3⁺Treg proportions in the face of an allergenic challenge in both the lung and draining LN.

### Example 2

### Materials and Methods

### Mice

Female BALB/c mice aged 7-8 weeks were obtained from Laboratory Animal Services (Adelaide, South Australia) and housed in the Alfred Medical Research and Education Precinct animal facility. All experimental protocols were approved by the precinct Animal Ethics Committee.

### Bermuda Grass Pollen

BGP was purchased from Greer Laboratories Inc. (Lenoir, N.C., USA) as dry, non-defatted pollen, and 1 g of pollen extracted in 5 ml of 1 mM NH₄HCO₃ overnight at 4° C. on a rotating wheel. After centrifugation, the supernatant was dialyzed against PBS overnight, filtered through a 0.2-μm filter, and the protein content determined using the Bio-Rad Microassay (Bio-Rad, USA).

### Particle Preparation, Particle Instillation and Immunizations

Polybead carboxylate microspheres (0.05 μm and 0.45 μm; Polysciences Inc. Warrington, Pa. USA #15913 and #09836, respectively) were glycine-coated as described previously (Fifis et al. 2004, J Immunol 173:3148-3154). In certain experiments mice received FITC-labelled particles (0.04 μm and 0.5 μm, Invitrogen-Molecular Probes, Carlsbad Calif., #F8795 and #F8813, respectively); pilot experiments indicated that the effects of unlabelled versus FITC-labelled particles were indistinguishable (data not shown). To determine particle effects on allergic airway inflammation, mice received saline (control) or particles (20 μg/50 μl) intratracheally (i.t.) (Hardy et al. 2003, supra) on d0 and d2. Mice were sensitised i.p. with saline or OVA (50 μg; Sigma-Aldrich) adsorbed to aluminium hydroxide on d12 and d22. Mice were challenged i.t. with saline or OVA (25 μg) on d32, d34, d37 and d39 as described previously (Hardy et al. 2003, supra). In certain experiments mice received FITC-labelled OVA for the final (4^{th}) challenge, or were sensitised and challenged with BGP. Mice were killed 24 hours after the final challenge.

### Tissue Sampling and Cell Isolation

Collection and preparation of blood, lung-draining LN, and BAL were as described previously (Hardy et al. 2003, supra). Viable LN and BAL cells were counted in a haemocytometer. For differentials, BAL cytospots were Giemsa-stained (Merck, Kilsyth Victoria) and ≧200 cells identified by morphological criteria. Tissue digestion was performed as described previously (Vremec et al. 1992, supra) with modifications. The right ventricle was perfused with 5 ml Ca²⁺/Mg²⁺-free HBSS (Invitrogen #14175095) with 0.01 M EDTA, pH 7.2. Lung-draining LN were minced with a scalpel blade, while lung tissue was chopped with a tissue chopper (Mickle Laboratory Engineering Co. Ltd, Gomshall, Surrey, UK). Tissue fragments were digested in collagenase type III (1 mg/ml; Worthington, Lakewood, N.J., USA) and DNase type I (0.025 mg/ml; Roche Diagnostics, Sydney NSW #1284932) at 25° C. mixing continuously for 45 minutes (LN) or 1 hour (lung). The reaction was stopped by adding one 10^{th} volume of EDTA and 3% FCS and mixing for 5 minutes. The cell suspension was filtered through a 70 □m cell strainer (BD Falcon), red cells lysed, and washed in staining buffer [3% FCS, 3% pooled normal mouse serum, 5 mM EDTA (pH 7.2) and 0.1% Na-Azide in Ca²⁺/Mg²⁺-free HBSS]. Viable cells were counted in a haemocytometer.

### Flow Cytometry

Non-specific FcR binding was blocked by incubating cells in CD16/CD32 block (BD Biosciences, San Jose, Calif., USA). Cells (0.5-1×10⁶) were stained on ice for 20 minutes with combinations of the following antibodies/conjugates (all BD unless noted): CD11b-PE, CD11c-APC, CD40-biotin, MHCII-PE and MHCII-biotin (AMS 32.1) and streptavidin-PerCP. Appropriate isotype control antibodies were used. All dilutions were in staining buffer (see above). Acquisition was on a FACSCalibur™ (BD), and analysis performed on FlowJo (Tree Star, Ashland, Oreg., USA).

### Cytokine ELISPOT

IL-4, IL-5, and IL-13 ELISPOT were performed as described previously (Hardy et al. 2006, supra). IFN-γ ELISPOT was performed using AN18 capture and R4-6A2 biotinylated detection antibodies (Mabtech, Mossman, NSW Australia; #3321-3-1000 and #3321-6-1000) and hydrophobic membrane plates (Millipore #MAIPS4510).

### OVA-Specific IgE ELISA

OVA-specific IgE was detected as described previously (Hardy et al. 2003, supra).

### Quantitation of Airway Mucus Production

Formalin-fixed paraffin embedded lung sections were stained with periodic acid-Schiff (PAS) reagent. The number of PAS-positive cells per small bronchiole (approx. basement membrane circumference 0.5 mm) were counted. Six to ten airways were counted per mouse.

### Statistical Analysis

Statistics were analysed using SPSS 15.0.1 software. Data were analysed for normality, and log-transformed as necessary prior to analysis by independent samples t-Test, ANOVA or two-way ANOVA with Tukey's HSD post-hoc analysis, as appropriate. Differences were considered statistically significant at p Group sizes are indicated in the Figure legends. All values are mean±s.e.m.

### Results

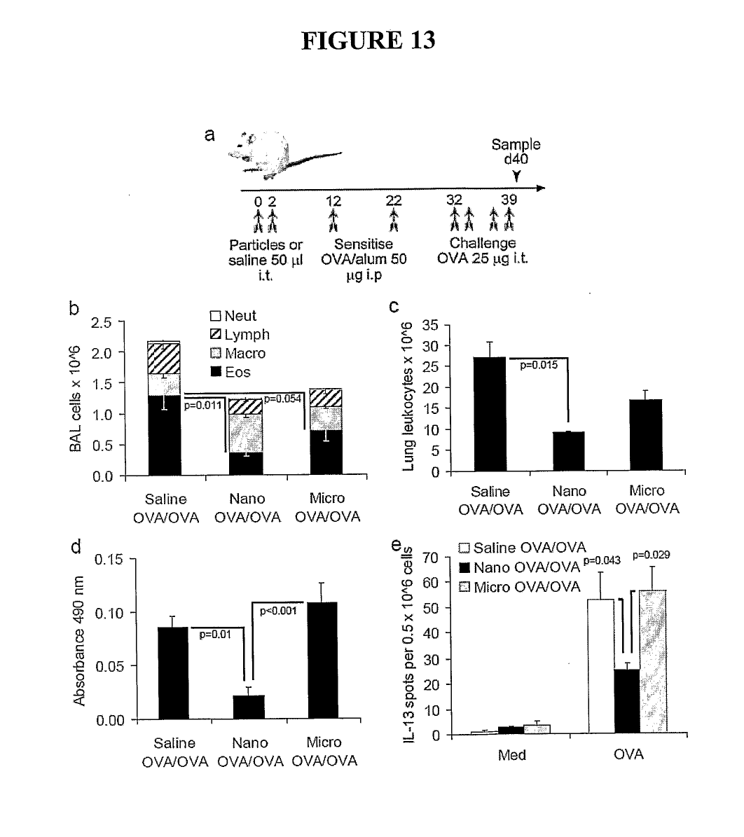

Previous findings suggesting that ultrafine particles have superior ability to promote acute airway inflammation relative to fine particles were based on analyses performed hours post-particle instillation ((Brown et al. 2001, supra; Kaewamatawong et al. 2005, supra; Renwick et al. 2004, supra). However, the long-term effects of ultrafine particles on lung immune cells, particularly particles devoid of potentially toxic (Borm et al. 2006, Part Fibre Toxicol 3:11; Oberdorster et al. 2005, Environ Health Perspect 113:823-839) or pro-inflammatory chemicals (e.g. transition metals (Wilson et al. 2002, Toxicol. Appl. Pharmacol. 184:172-179) are unknown. The long-term consequences of pulmonary exposure to chemically inert glycine-coated 50 nm (nanoparticles) and 500 nm (microparticles) polystyrene particles (Fifis 2004, supra; Mottram et al. 2007, Molecule. Pharm. 4:73-84) were tested in a well-established model of acute allergic airway inflammation (Hardy et al. 2003, supra; Hardy et al. 2006, supra). In the following investigations, particles were instilled prior to allergen sensitisation/challenge, and not coadministered with allergen, in contrast to other studies (Alessandrini et al. 2006, J. Allergy. Clin. Immunol. 26:2706-2713; deHaar et al. 2006, Clin. Exp. Allergy 36:1469-1479). Mice received 50 or 500 nm particles intratracheally 10-12 days prior to systemic allergen sensitisation, with allergen challenges commencing d32 (FIG. 13A). Pilot studies showed that 31 days after particle instillation into naive mice the bronchoalveolar lavage (BAL) cell numbers were virtually identical to saline treated controls (data not shown). However, unexpectedly, both nano- and microparticle pre-treatment dramatically decreased inflammation of the airways (largely due to a marked decrease in airway eosinophil numbers) and parenchymal lung tissue in response to allergen challenge compared to mice which did not receive particles, with 50 nm particles showing the strongest activity (FIGS. 13B & 13C). Importantly, only nanoparticles further inhibited the production of key molecules associated with induction and maintenance of allergic asthma symptoms: allergen-specific IgE (FIG. 13D) and IL-13 (FIG. 13E), which together critically regulate acute allergic reactions, airway hyperreactivity, airway mucus production and IgE isotype switching.

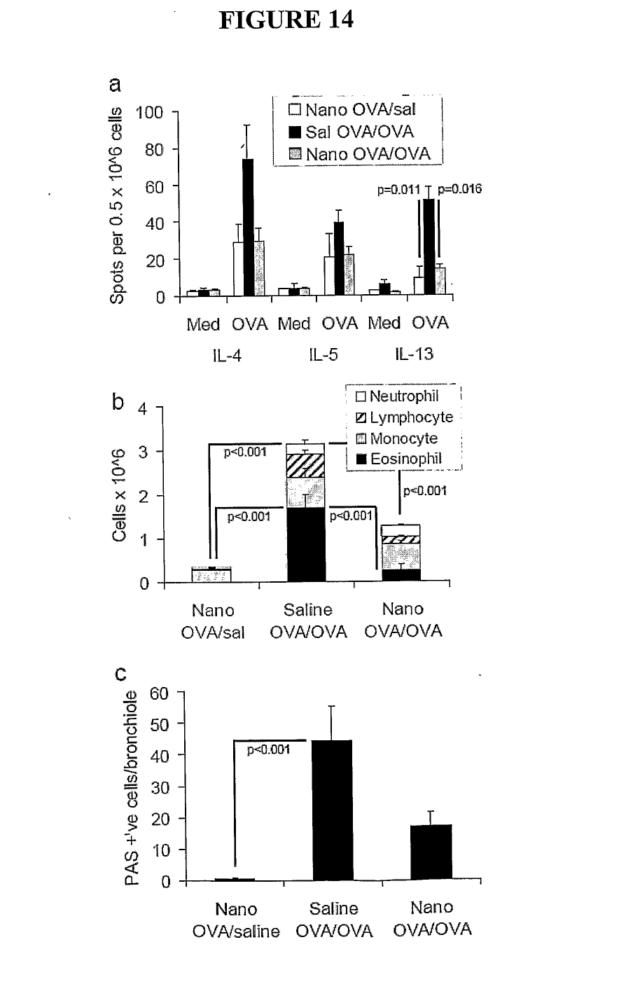

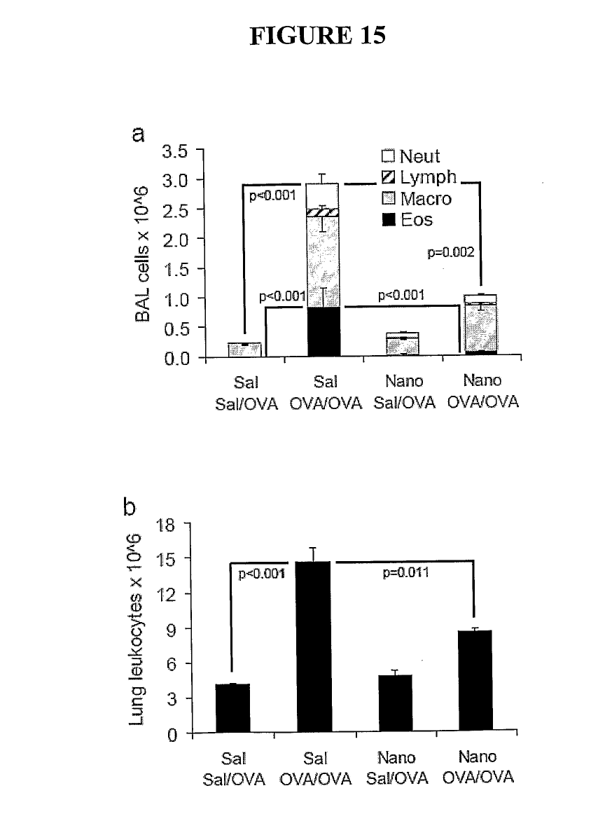

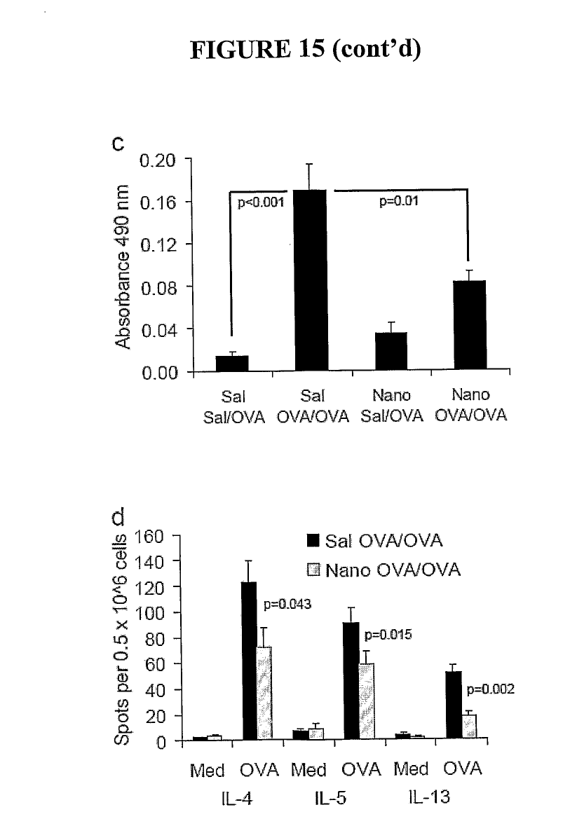

To investigate further 50 nm particle pre-treatment affected pulmonary Th2 immunity by interfering with peripheral antigen sensitisation, prior to the local challenge with antigen, the effect of 50 nm particles in mice which were sensitised but not challenged was investigated. Allergen-specific IgE was induced at comparable concentration in ovalbumin (OVA)-sensitised mice, whether pre-treated with 50 nm particles or not (0.11±0.36 OD units versus 0.08±0.002, saline versus 50 nm, respectively). Furthermore, in the absence of allergen challenge, 50 nm particles did not prevent the induction of Th2 cytokine producing cells in the lung-draining lymph node (LN) (nano/OVA/sal) (FIG. 14A) or spleen (data not shown), indicating that they did not impair allergen sensitisation. Additional experiments were performed to determine the effect of nanoparticles in mice which did not have allergic airway inflammation [ie saline sensitised and OVA challenged (nano/sal/OVA)] and to study their effect on the key Th2 cytokines IL-4 and IL-5. Instillation of 50 nm particles prior to OVA sensitisation/challenge significantly inhibited eosinophilic airway inflammation (FIG. 15A), lung inflammatory cells (FIG. 15B) and serum allergen-specific IgE (FIG. 15C) compared to the sal/OVA/OVA group. Furthermore, 50 nm particles caused a >2-fold reduction in the frequency of airway mucus-secreting cells (FIG. 14C). Importantly, nanoparticles did not cause airway or lung inflammation, or IgE production, in the absence of allergen sensitisation (sal/sal/OVA versus nano/sal/OVA, FIGS. 15A-C), or airway inflammation or mucus production in the absence of allergen challenge (nano/OVA/sal, FIGS. 14B & 1C). Thus, nanoparticles do not exert their effects at the systemic priming stage, but rather impair efficient induction of pulmonary inflammation at the allergen challenge phase.

Strikingly, 50 nm particle instillation prior to sensitisation/challenge (nano/OVA/OVA) caused a generalised inhibition of key Th2 cytokine production (IL-4, IL-5 and IL-13) in the lung-draining LN (FIG. 15D). However, this prevention of local Th2 immunity was not counter-regulated by nanoparticle-driven induction of Th1 cells in the draining LN (Cohn et al. 1999 J. Exp. Med. 190:1309-1318; Huang et al. 2001, J. Immunol 166:207-217) as the frequency of IFN-γ producing cells in both the nano/OVA/OVA and saline/OVA/OVA groups was comparable and low (<10 per 0.5×10⁶ cells). Indeed, nanoparticles decreased the frequency of Th2 cytokine-producing cells to the level seen in mice which were sensitised but not challenged (nano/OVA/sal versus nano/OVA/OVA, FIG. 14A), suggesting the triggering of a homeostatic mechanism. The decreased production of IL-4, IL-5 and IL-13 correlates with the decreased allergen-specific IgE concentrations, airway eosinophilia and mucus hypersecretion, respectively (Lebman & Coffman 1988, J. Exp. Med. 168:853-862; Foster et al. 1996, J. Exp. Med. 183:195-201). This dampening of Th2 immunity specifically in the lung is consistent with local particle-induced immunomodulatory mechanisms.

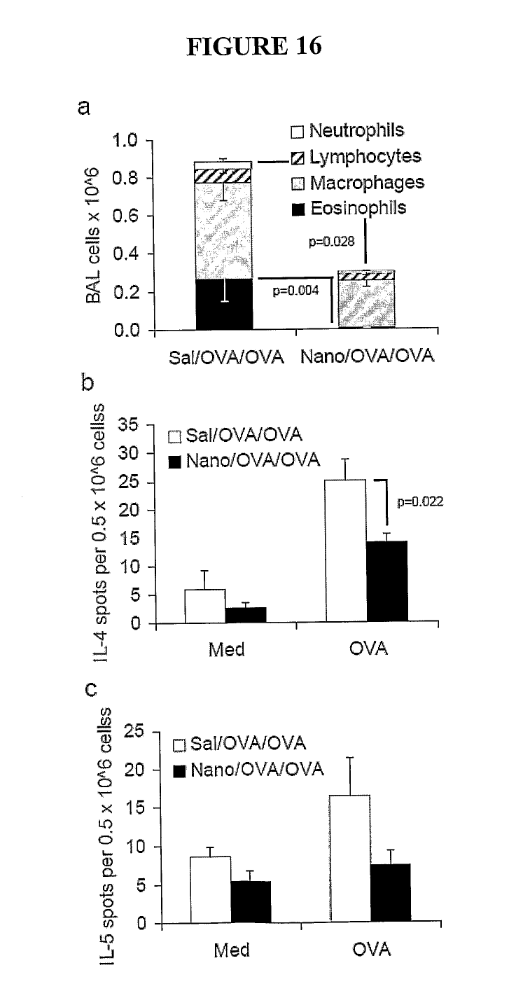

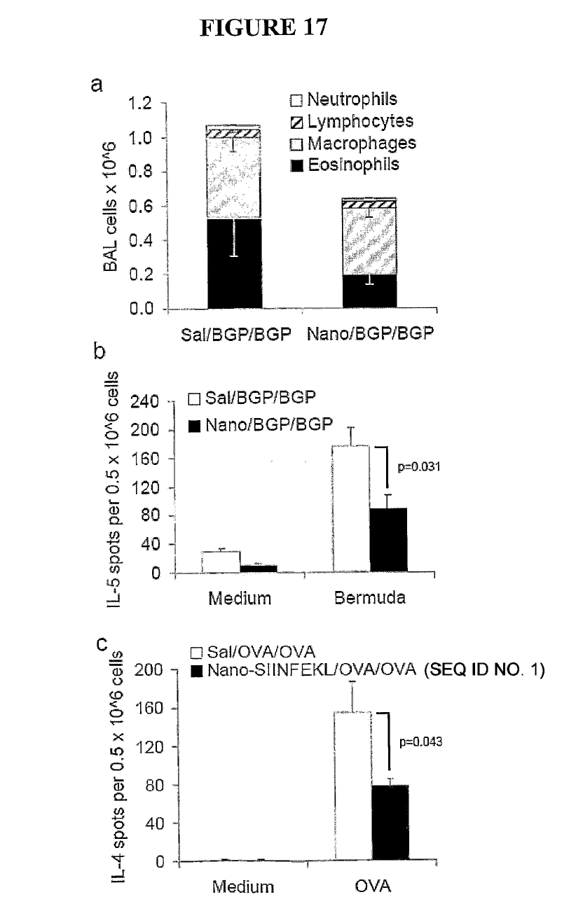

It was also confirmed that nanoparticle protective effects were not unique to our OVA-induced model of allergic airway inflammation in the BALB/c (H-2^{b}) strain. Nanoparticle pre-treatment of C57BL/6 (H-2^{d}) mice markedly inhibited eosinophilic airway inflammation and Th2 cytokine production in the draining LN (FIG. 16). To additionally test whether the effect was allergen-dependent, in a separate experiment BALB/c mice received nanoparticles prior to sensitisation/challenge with the clinically-relevant seasonal allergen Bermuda grass pollen (BGP) (Couch grass). There was a >2-fold reduction in airway eosinophils and frequency of IL-5-producing draining LN cells (FIGS. 17A & 17B). Nanoparticles conjugated to an irrelevant peptide (the H-2^{b}-restricted MHC Class I OVA epitope SIINFEKL) similarly inhibit IL-4 production in H-2^{d} BALB/c mice (FIG. 17C). Fluorescently-labelled nanoparticles and unlabelled nanoparticles also induced identical inhibition of allergic airway inflammation (data not shown). Together, these data show that nanoparticles have the capacity to prevent allergic airway inflammation independent of the allergen used or genetic background, and that there will be a degree of flexibility for particle surface coating with amino acids or peptides.

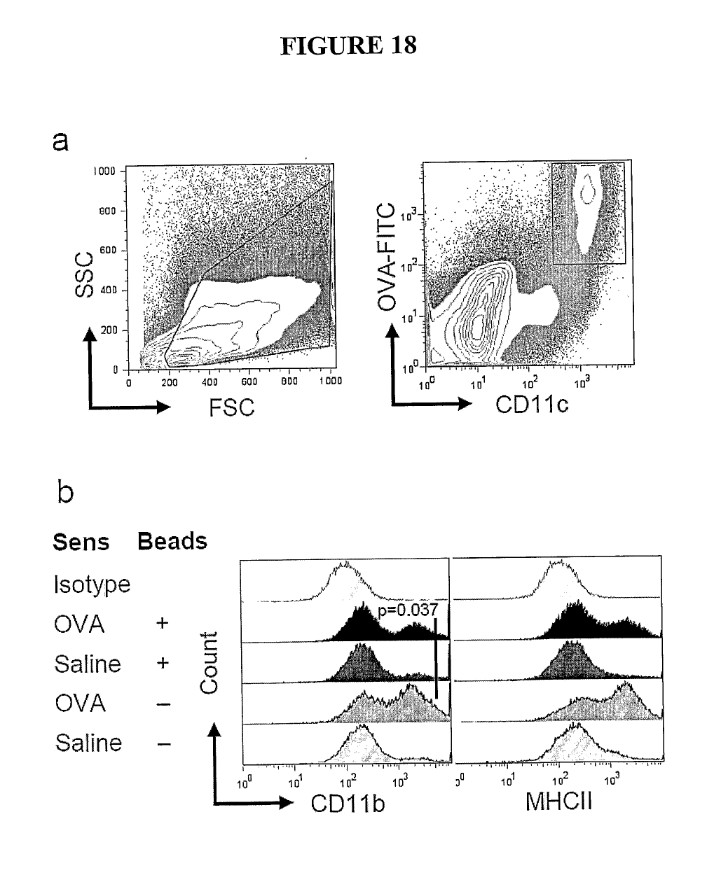

Pulmonary dendritic cells (DC) play a critical role in the generation of allergic immune responses (Lambrecht 2003, supra). To study whether 50 nm particle treatment affected allergen uptake by DC, allergen-sensitised mice were challenged with FITC-labelled allergen to allow the subsequent identification of allergen-laden DC (OVA-FITC⁺CD11c⁺ cells)²⁸ (FIG. 18A); pilot experiments showed that lung FITC⁺CD11c⁺ cells consisted almost entirely of cells with macrophage/DC morphology. The absolute number of allergen-laden lung DC (OVA-FITC⁺CD11c⁺) was increased approximately 2-fold in sensitised/challenged mice (0.34±0.2×10⁶ versus 0.64±0.04×10⁶, saline versus OVA challenge, p=0.002), and this was marginally prevented by particle instillation (0.55±0.04×10⁶). More profoundly, nanoparticle treatment decreased by approximately one third the proportion of lung OVA-FITC⁺CD11c⁺ cells that co-expressed MHCII or CD11b at high density (FIG. 18B). In the lung, CD11b expression identifies two major DC subsets (Wikstrom et al. 2007 supra; Sung et al. 2006, J. Immunol. 176:2161-2172), with the CD11c⁺MHCII^{hi}CD11b^{hi} population being the dominant chemokine-producing DC with strong T cell stimulatory capacity (Beaty et al. 2007, J. Immunol. 178:1882-1895). OVA-FITC⁺CD11c⁺ cells in the lung of control non-sensitised mice had a relatively immature phenotype as judged by low MHCII expression, and were predominantly CD11b^{low}. This was not changed by 50 nm particle treatment. CD40 expression by lung OVA-FITC⁺CD11c⁺ cells in sensitised/challenged mice was increased approximately 2-fold compared to non-sensitised controls, and this was not altered by particle instillation. In contrast, when DC were identified by analysis of ‘total’ MHCII^{hi}CD11c⁺ cells (ie including allergen-laden and non-allergen-laden cells), 50 nm particle treatment of sensitised/challenged mice did not decrease the frequency of CD11b^{hi} cells, while their frequency in non-sensitised mice was increased. Thus, analysis of allergen-laden cells (OVA-FITC⁺CD11c⁺) revealed dramatic particle-induced differences in CD11b expressing populations which are not detected using general (MHCII^{hi}CD11c⁺) DC gating. In contrast to the lung, neither 50 nm particle treatment nor inflammatory status altered CD11b or MHCII expression by FITC⁺CD11c⁺ cells in the draining LN. Therefore, chemically inert nanoparticles decrease the frequency of a major stimulatory DC subset amongst allergen-laden DC in the lung, without affecting draining LN DC populations, suggesting additional mechanisms could operate to further down-regulate Th2 immunity in the LN.

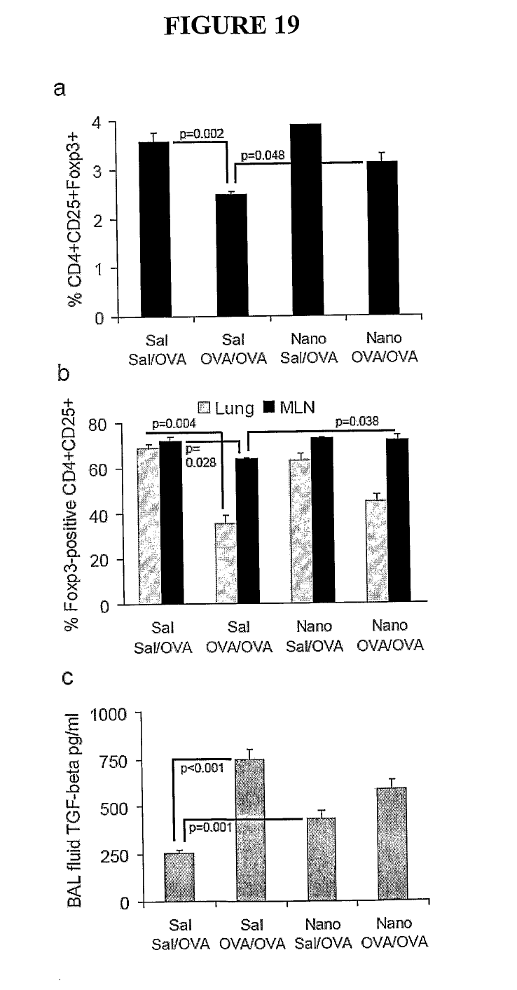

Regulatory CD4⁺CD25⁺T cells expressing high levels of Foxp3 (Treg) play a central role in the regulation of allergic asthma and downregulation of Th2 immunity (Hawrylowicz & O'Garra 2005, Nat. Rev. Immunol. 5:271-2831; Kearley et al. 2005 supra; Strickland et al. 2006, supra). The frequency of lung CD4⁺CD25⁺Foxp3⁺ cells in naive mice was found to be increased 2-fold by 50 nm but not 500 nm particles at d31 post-instillation (0.6±0.1% versus 1.2±0.2%, saline versus 50 nm, respectively, p=0.031). Furthermore, in mice with allergic airway inflammation the frequency of draining LN CD4⁺CD25⁺Foxp3⁺ cells was decreased by one third (sal/sal/OVA versus sal/OVA/OVA, FIG. 19A), and this was partially prevented by prior 50 nm particle treatment (nano/OVA/OVA); a similar but less marked trend was observed in the lung (data not shown). A different analysis showed that 58±2% of CD4⁺CD25⁺ draining LN cells expressed Foxp3 in mice which were sensitised and challenged but did not receive 50 nm particles, while 50 nm particle treatment prior to sensitisation/challenge increased this value to 71±1% (nano/OVA/OVA), similar to the value in mice without allergic airway inflammation, regardless of whether they were particle treated or not (FIG. 19B). Similarly in the lung, the frequency of Foxp3⁺ cells within the CD4⁺CD25⁺ population was decreased 2-fold in mice with allergic airway inflammation, and this was partially prevented by nanoparticles (FIG. 19B). Also assessed was whether particle instillation by itself, in otherwise unmanipulated mice, affected the frequency of Foxp3⁺ cells within the CD4⁺CD25⁺ population 31 days post-instillation. Naïve mice treated with 50 nm, but not 500 nm particles had a marginally (non significant) increased frequency of Foxp3⁺ cells within the CD4⁺CD25⁺ population in the LN and the lung compared to non particle-treated mice (data not shown). Treg can regulate inflammation, including allergy and asthma, via production of the cytokines TGF-β and/or IL-10, and TGF-β is required for maintenance of Treg suppressive function (Marie et al. 2005, J. Exp. Med. 201:1061-1067). BAL fluid TGF-β concentrations were increased in sensitised/challenged mice compared to non-sensitised controls, typical of mice with allergic airway inflammation (Alcorn et al. 2007, Am J Respir Grit Care Med 176:974-982), although this was not increased further by 50 nm particles (FIG. 4C). There was no difference in BAL fluid IL-10 concentrations in any of the groups (approximately 3500 pg/ml regardless of allergic status or particle treatment, data not shown). Together, these data show that 50 nm particles increased Treg frequencies at a time immediately prior to allergen challenge (d31), and this was sustained post-challenge. Previous studies have shown a correlation between elevated pulmonary Treg numbers and suppression of airway inflammation (Strickland et al. 2006, supra). Therefore, these data show that particle-induced maintenance of Treg frequencies in the lung and draining LN upon allergen challenge are consistent with the observed dampening of allergic airway inflammation resulting from 50 nm particle treatment.

Collectively these findings demonstrate that inert nanoparticles have the capacity to generate a lung state resistant to allergic airway inflammation. Importantly, this feature is strictly size-dependent, with nanoparticles but not microparticles being able to turn off Th2 immunity, characterised by a decrease in stimulatory DC in the lung and an increased proportion of lung and draining LN residing Treg. Since 50 nm, but not 500 nm particles prevented the full spectrum of innate and adaptive inflammatory responses elicited upon allergen challenge in atopic (sensitised) animals, size clearly pays a critical role in enabling this novel biological activity.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Akbari, O., G. J. Freeman, E. H. Meyer, E. A. Greenfield, T. T. Chang, A. H. Sharpe, G. Berry, R. H. DeKruyff, and D. T. Umetsu. 2002. Antigen-specific regulatory T cells develop via the ICOS-ICOS-ligand pathway and inhibit allergen-induced airway hyperreactivity. Nat Med 9:1024-1032
Akbari, O., R. H. DeKruyff, and D. T. Umetsu. 2001. Pulmonary dendritic cells producing IL-10 mediate tolerance induced by respiratory exposure to antigen. Nat Immunol 2:725-731.
Akdis, C. A., T. Blesken, M. Akdis, B. Wuthrich, and K. Blaser. 1998. Role of interleukin 10 in specific immunotherapy. J Clin Invest 102:98-106.
Alcorn, J. F. et al. Transforming growth factor-beta1 suppresses airway hyperresponsiveness in allergic airway disease. Am J Respir Crit Care Med 176:974-982 (2007)
Beaty, S. R., Rose, C. E., Jr. & Sung, S. S. Diverse and potent chemokine production by lung CD11bhigh dendritic cells in homeostasis and in allergic lung inflammation. J Immunol 178:1882-1895 (2007)
Bernstein, J. A., N. Alexis, C. Barnes, I. L. Bernstein, A. Nel, D. Peden, D. Diaz-Sanchez, S. M. Tarlo, and P. B. Williams. 2004. Health effects of air pollution. J Allergy Clin Immunol 114:1116-1123.
Borm, P. J. et al. The potential risks of nanomaterials: a review carried out for ECETOC. Part Fibre Toxicol 3:11 (2006)
Brown, D. M., K. Donaldson, P. J. Borm et al. 2004. Calcium and ROS-mediated activation of transcription factors and TNF-alpha cytokine gene expression in macrophages exposed to ultrafine particles. Am J Physiol Lung Cell Mol Physiol 286:L344.
Brown, D. M., M. R. Wilson, W. MacNee, V. Stone, and K. Donaldson. 2001. Size-dependent proinflammatory effects of ultrafine polystyrene particles: a role for surface area and oxidative stress in the enhanced activity of ultrafines. Toxicol Appl Pharmacol 175:191-199.
Brunekreef, B., and S. T. Holgate. 2002. Air pollution and health. Lancet 360:1233-1242.
Cohn, L., Homer, R. J., Niu, N. & Bottomly, K. T helper 1 cells and interferon gamma regulate allergic airway inflammation and mucus production. J Exp Med 190:1309-1318 (1999)
Constant, S. L., J. L. Brogdon, D. A. Piggott, C. A. Herrick, I. Visintin, N. H. Ruddle, and K. Bottomly. 2002. Resident lung antigen-presenting cells have the capacity to promote Th2 T cell differentiation in situ. J Clin Invest 110:1441-1448.
Dailey, L. A., N. Jekel, L. Fink, T. Gessler, T. Schmehl, M. Wittmar, T. Kissel, and W. Seeger. 2006. Investigation of the proinflammatory potential of biodegradable nanoparticle drug delivery systems in the lung. Toxicol Appl Pharmacol 215:100.
de Haar, C., Hassing, I., Bol, M., Bleumink, R. & Pieters, R. Ultrafine but not fine particulate matter causes airway inflammation and allergic airway sensitization to co-administered antigen in mice. Clin Exp Allergy 36:1469-1479 (2006)
de Haar, C., I. Hassing, M. Bol, R. Bleumink, and R. Pieters. 2005. Ultrafine carbon black particles cause early airway inflammation and have adjuvant activity in a mouse allergic airway disease model. Toxicol Sci 87:409-418.
Fifis, T., A. Gamvrellis, B. Crimeen-Irwin, G. A. Pietersz, J. Li, P. L. Mottram, I. F. McKenzie, and M. Plebanski. 2004. Size-dependent immunogenicity: therapeutic and protective properties of nano-vaccines against tumors. J Immunol 173:3148-3154.
Foged, C., B. Brodin, S. Frokjaer, and A. Sundblad. 2005. Particle size and surface charge affect particle uptake by human dendritic cells in an in vitro model. Int J Pharm 298:315-322.
Foster, P. S., Hogan, S. P., Ramsay, A. J., Matthaei, K. I. & Young, I. G. Interleukin 5 deficiency abolishes eosinophilia, airways hyperreactivity, and lung damage in a mouse asthma model. J Exp Med 183:195-201 (1996)
Gardner, L. M., F. C. Thien, J. A. Douglass, J. M. Rolland, and R. E. O'Hehir. 2004. Induction of T ‘regulatory’ cells by standardized house dust mite immunotherapy: an increase in CD4+CD25+ interleukin-10+T cells expressing peripheral tissue trafficking markers. Clin Exp Allergy 34:1209-1219.
Gauderman, W. J., H. Vora, R. McConnell, K. Berhane, F. Gilliland, D. Thomas, F. Lurmann, E. Avol, N. Kunzli, M. Jerrett, and J. Peters. 2007. Effect of exposure to traffic on lung development from 10 to 18 years of age: a cohort study. Lancet 369:571-577.
Hansen, G., J. J. McIntire, V. P. Yeung, G. Berry, G. J. Thorbecke, L. Chen, R. H. DeKruyff, and D. T. Umetsu. 2000. CD4(+) T helper cells engineered to produce latent TGF-beta1 reverse allergen-induced airway hyperreactivity and inflammation. J Clin Invest 105:61-70.
Hardy, C. L., E. O'Connor A, J. Yao, K. Sebire, D. M. de Kretser, J. M. Rolland, G. P. Anderson, D. J. Phillips, and E. O'Hehir R. 2006. Follistatin is a candidate endogenous negative regulator of activin A in experimental allergic asthma. Clin Exp Allergy 36:941-950.
Hardy, C. L., L. Kenins, A. C. Drew, J. M. Rolland, and R. E. O'Hehir. 2003. Characterization of a mouse model of allergy to a major occupational latex glove allergen Hey b 5. Am J Respir Crit Care Med 167:1393-1399.
Harford, A. J., K. O'Halloran, and P. F. Wright. 2005. The effects of in vitro pesticide exposures on the phagocytic function of four native Australian freshwater fish. Aquat Toxicol 75:330.
Hawrylowicz, C. M. & O'Garra, A. Potential role of interleukin-10-secreting regulatory T cells in allergy and asthma. Nat Rev Immunol 5:271-283 (2005)
Heinrich, J., and H. E. Wichmann. 2004. Traffic related pollutants in Europe and their effect on allergic disease. Curr Opin Allergy Clin Immunol 4:341-348.
Huang, T. J. et al. Allergen-specific Th1 cells counteract efferent Th2 cell-dependent bronchial hyperresponsiveness and eosinophilic inflammation partly via IFN-gamma. J Immunol 166:207-217 (2001)
Huh, J. C., D. H. Strickland, F. L. Jahnsen, D. J. Turner, J. A. Thomas, S. Napoli, I. Tobagus, P. A. Stumbles, P. D. Sly, and P. G. Holt. 2003. Bidirectional interactions between antigen-bearing respiratory tract dendritic cells (DCs) and T cells precede the late phase reaction in experimental asthma: DC activation occurs in the airway mucosa but not in the lung parenchyma. J Exp Med 198:19-30.
Humbles, A. A., C. M. Lloyd, S. J. McMillan et al. 2004. A critical role for eosinophils in allergic airways remodeling. Science 305:1776.
Kaewamatawong, T., N. Kawamura, M. Okajima, M. Sawada, T. Morita, and A. Shimada. 2005. Acute pulmonary toxicity caused by exposure to colloidal silica: particle size dependent pathological changes in mice. Toxicol Pathol 33:745-751.
Kearley, J., J. E. Barker, D. S. Robinson, and C. M. Lloyd. 2005. Resolution of airway inflammation and hyperreactivity after in vivo transfer of CD4+CD25+ regulatory T cells is interleukin 10 dependent. J Exp Med 202:1539-1547.
Kolb, M., P. J. Margetts, D. C. Anthony et al. 2001. Transient expression of IL-1beta induces acute lung injury and chronic repair leading to pulmonary fibrosis. J Clin Invest 107:1529.
Lambrecht, B. N., and H. Hammad. 2003. Taking our breath away: dendritic cells in the pathogenesis of asthma. Nat Rev Immunol 3:994-1003.
Lambrecht, B. N., R. A. Pauwels, and B. Fazekas De St Groth. 2000. Induction of rapid T cell activation, division, and recirculation by intratracheal injection of dendritic cells in a TCR transgenic model. J Immunol 164:2937-2946.
Lebman, D. A. & Coffman, R. L. Interleukin 4 causes isotype switching to IgE in T cell-stimulated clonal B cell cultures. J Exp Med 168:853-862 (1988)
Marie, J. C., Letterio, J. J., Gavin, M. & Rudensky, A. Y. TGF-beta1 maintains suppressor function and Foxp3 expression in CD4+CD25+ regulatory T cells. J Exp Med 201:1061-1067 (2005)
Martin-Fontecha, A., S. Sebastiani, U. E. Hopken, M. Uguccioni, M. Lipp, A. Lanzavecchia, and F. Sallusto. 2003. Regulation of dendritic cell migration to the draining lymph node: impact on T lymphocyte traffic and priming. J Exp Med 198:615-621.
McWilliam, A. S., S. Napoli, A. M. Marsh, F. L. Pemper, D. J. Nelson, C. L. Pimm, P. A. Stumbles, T. N. Wells, and P. G. Holt. 1996. Dendritic cells are recruited into the airway epithelium during the inflammatory response to a broad spectrum of stimuli. J Exp Med 184:2429-2432.
Mottram, P. L. et al. Type 1 and 2 immunity following vaccination is influenced by nanoparticle size: formulation of a model vaccine for respiratory syncytial virus. Mol Pharm 4, 73-84 (2007) Alessandrini, F. et al. Effects of ultrafine carbon particle inhalation on allergic inflammation of the lung. J Allergy Clin Immunol 117:824-830 (2006)
Nakao, A., S. Miike, M. Hatano, K. Okumura, T. Tokuhisa, C. Ra, and I. Iwamoto. 2000. Blockade of transforming growth factor beta/Smad signaling in T cells by overexpression of Smad7 enhances antigen-induced airway inflammation and airway reactivity. J Exp Med 192:151-158.
Oberdorster, G. 2001. Pulmonary effects of inhaled ultrafine particles. Int Arch Occup Environ Health 74:1-8.
Oberdorster, G., Oberdorster, E. & Oberdorster, J. Nanotoxicology: an emerging discipline evolving from studies of ultrafine particles. Environ Health Perspect 113:823-839 (2005)
Oh, J. W., C. M. Seroogy, E. H. Meyer, O. Akbari, G. Berry, C. G. Fathman, R. H. Dekruyff, and D. T. Umetsu. 2002. CD4 T-helper cells engineered to produce IL-10 prevent allergen-induced airway hyperreactivity and inflammation. J Allergy Clin Immunol 110:460-468.
Ostroukhova, M., C. Seguin-Devaux, T. B. Oriss, B. Dixon-McCarthy, L. Yang, B. T. Ameredes, T. E. Corcoran, and A. Ray. 2004. Tolerance induced by inhaled antigen involves CD4(+) T cells expressing membrane-bound TGF-beta and FOXP3. J Clin Invest 114:28-38.
Peters, A., H. E. Wichmami, T. Tuch, J. Heinrich, and J. Heyder. 1997. Respiratory effects are associated with the number of ultrafine particles. Am J Respir Crit Care Med 155:1376-1383.
Plebanski, M., and S. S. Burtles. 1994. In vitro primary responses of human T cells to soluble protein antigens. J Immunol Methods 170:15.
Plebanski, M., C. J. Elson, and W. D. Billington. 1992. Dependency on interleukin-1 of primary human in vitro T cell responses to soluble antigens. Eur J Immunol 22:2353.
Plebanski, M., M. Saunders, S. S. Burtles, S. Crowe, and D. C. Hooper. 1992. Primary and secondary human in vitro T-cell responses to soluble antigens are mediated by subsets bearing different CD45 isoforms. Immunology 75:86.
Pouniotis, D. S., M. Plebanski, V. Apostolopoulos, and C. F. McDonald. 2006. Alveolar macrophage function is altered in patients with lung cancer. Clin Exp Immunol 143:363-372.
Priestly, B. G., and A. J. Harford. 2007. The Human Health Risk Assessment (HHRA) of nanomaterials. Edward Elgar publications, Cheltenham.
Randolph, G. J., V. Angeli, and M. A. Swartz. 2005. Dendritic-cell trafficking to lymph nodes through lymphatic vessels. Nat Rev Immunol 5:617.
Renwick, L. C., D. Brown, A. Clouter, and K. Donaldson. 2004. Increased inflammation and altered macrophage chemotactic responses caused by two ultrafine particle types. Occup Environ Med 61:442-447.
Scheerlinck, J. P., S. Gloster, A. Gamvrellis, P. L. Mottram, and M. Plebanski. 2006. Systemic immune responses in sheep, induced by a novel nano-bead adjuvant. Vaccine 24:1124-1131.
Schon-Hegrad, M. A., J. Oliver, P. G. McMenamin, and P. G. Holt. 1991. Studies on the density, distribution, and surface phenotype of intraepithelial class II major histocompatibility complex antigen (Ia)-bearing dendritic cells (DC) in the conducting airways. J Exp Med 173:1345-1356.
Senior, R. and Griffin, G. (1980). Chemotactic activity of elastin-derived peptides. J Clin Invest, 66(4):859-862
Shapiro, 1998
Strickland, D. H., P. A. Stumbles, G. R. Zosky, L. S. Subrata, J. A. Thomas, D. J. Turner, P. D. Sly, and P. G. Holt. 2006. Reversal of airway hyperresponsiveness by induction of airway mucosal CD4+CD25+ regulatory T cells. J Exp Med 203:2649-2660.
Stumbles, P. A., D. H. Strickland, C. L. Pimm, S. F. Proksch, A. M. Marsh, A. S. McWilliam, A. Bosco, I. Tobagus, J. A. Thomas, S. Napoli, A. E. Proudfoot, T. N. Wells, and P. G. Holt. 2001. Regulation of dendritic cell recruitment into resting and inflamed airway epithelium: use of alternative chemokine receptors as a function of inducing stimulus. J Immunol 167:228-234.
Stumbles, P. A., J. A. Thomas, C. L. Pimm, P. T. Lee, T. J. Venaille, S. Proksch, and P. G. Holt. 1998. Resting respiratory tract dendritic cells preferentially stimulate T helper cell type 2 (Th2) responses and require obligatory cytokine signals for induction of Th1 immunity. J Exp Med 188:2019-2031.
Sung, S. S. et al. A major lung CD103 (alphaE)-beta7 integrin-positive epithelial dendritic cell population expressing Langerin and tight junction proteins. J Immunol 176:2161-2172 (2006)
Tang, C., J. M. Rolland, X. Li, C. Ward, R. Bish, and E. H. Walters. 1998. Alveolar macrophages from atopic asthmatics, but not atopic nonasthmatics, enhance interleukin-5 production by CD4+T cells. Am J Respir Crit Care Med 157:1120.
Tsitoura, D. C., R. H. DeKruyff, J. R. Lamb, and D. T. Umetsu. 1999. Intranasal exposure to protein antigen induces immunological tolerance mediated by functionally disabled CD4+T cells. J Immunol 163:2592-2600.
van den Berg, T. K., and G. Kraal. 2005. A function for the macrophage F4/80 molecule in tolerance induction. Trends Immunol 26:506-509.
van Rijt, L. S., S. Jung, A. Kleinj an, N. Vos, M. Willart, C. Duez, H. C. Hoogsteden, and B. N. Lambrecht. 2005. In vivo depletion of lung CD11c⁺ dendritic cells during allergen challenge abrogates the characteristic features of asthma. J Exp Med 201:981-991.
Vermaelen, K., and R. Pauwels. 2003. Accelerated airway dendritic cell maturation, trafficking, and elimination in a mouse model of asthma. Am J Respir Cell Mol Biol 29:405-409.
Vermaelen, K., and R. Pauwels. 2005. Pulmonary dendritic cells. Am J Respir Crit Care Med 172:530-551.
Vermaelen, K. Y., I. Carro-Muino, B. N. Lambrecht, and R. A. Pauwels. 2001. Specific migratory dendritic cells rapidly transport antigen from the airways to the thoracic lymph nodes. J Exp Med 193:51-60.
von Garnier, C., L. Filgueira, M. Wikstrom, M. Smith, J. A. Thomas, D. H. Strickland, P. G. Holt, and P. A. Stumbles. 2005. Anatomical location determines the distribution and function of dendritic cells and other APCs in the respiratory tract. J Immunol 175:1609-1618.
von Klot, S., G. Wolke, T. Tuch, J. Heinrich, D. W. Dockery, J. Schwartz, W. G. Kreyling, H. E. Wichmann, and A. Peters. 2002. Increased asthma medication use in association with ambient fine and ultrafine particles. Eur Respir J 20:691-702.
Vremec, D., M. Zorbas, R. Scollay, D. J. Saunders, C. F. Ardavin, L. Wu, and K. Shortman. 1992. The surface phenotype of dendritic cells purified from mouse thymus and spleen: investigation of the CD8 expression by a subpopulation of dendritic cells. J Exp Med 176:47-58.
Wikstrom, M. E., and P. A. Stumbles. 2007. Mouse respiratory tract dendritic cell subsets and the immunological fate of inhaled antigens. Immunol Cell Biol 85:182-188.
Wilson, M. R., Lightbody, J. H., Donaldson, K., Sales, J. & Stone, V. Interactions between ultrafine particles and transition metals in vivo and in vitro. Toxicol Appl Pharmacol 184:172-179 (2002)
Woessner, J. F., Jr. 1961. The determination of hydroxyproline in tissue and protein samples containing small proportions of this imino acid. Arch Biochem Biophys 93:440.
Xia, W., C. E. Pinto, and R. L. Kradin. 1995. The antigen-presenting activities of Ia+ dendritic cells shift dynamically from lung to lymph node after an airway challenge with soluble antigen. J Exp Med 181:1275-1283.

1. A method for the therapeutic or prophylactic treatment of antigen-induced airway tissue inflammation in a mammal said method comprising contacting said airway tissue with an effective amount of an ultrafine particle, wherein said ultrafine particle induces or maintains non-inflammatory airway tissue homeostasis.

2-22. (canceled)

23. The method according to claim 1, wherein said ultrafine particle is inert.

24. The method according to claim 1, wherein said airway tissue is lung tissue.

25. The method according to claim 1, wherein said antigen is an allergen, pathogen, tobacco related particle, environmental particle, chemical or synthetic particle or an organism derived particle.

26. The method according to claim 25, wherein said allergen is a plant derived allergen, chemical pollutant, synthetic pollutant, airborne allergen or an organism derived allergen.

27. The method according to claim 26, wherein said allergen is pollen, dust or mite feces.

28. The method according to claim 25, wherein said pathogen is a virus, bacterium or parasite.

29. The method according to claim 28, wherein said virus is respiratory syncytial virus, rhinovirus, influenza virus, cytomegalovirus or parainfluenza virus.

30. The method according to claim 1, wherein said antigen induced airway inflammation is associated with airway hypersensitivity, infection, asthma, emphysema, COPD, acute respiratory distress syndrome, pneumonia, acute lung injury, lung fibrosis or bronchiectasis.

31. The method according to claim 1, wherein said ultrafine particle is 30 nm-70 nm.

32. The method according to claim 31, wherein said ultrafine particle is 35 nm-65 nm.

33. The method according to claim 32, wherein said ultrafine particle is 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm or 65 nm.

34. The method according to claim 33, wherein said ultrafine particle is 45 nm, 46 nm, 47 nm, 48 nm, 49 nm, 50 nm, 51 nm, 52 nm, 53 nm, 54 nm or 55 nm.

35. The method according to claim 1, wherein said ultrafine particle is formed from or coated with a polymer, inorganic material, metal or an organic material.

36. The method according to claim 35, wherein said inorganic material is ceramic or glass.

37. The method according to claim 35, wherein said polymer is biodegradable.

38. The method according to claim 35, wherein said polymer is a polystyrene, a polyacrylate, a polymethacrylate, a polyolefin, a polypropylene, a polyethylene, a polyfluorocarbon, a Teflon, a polyurethane, a polyamide, a polycarbonate or a polyether.

39. The method according to claim 37, wherein said biodegradable polymer is a biodegradable polyurethane, a biodegradable polyester or a biodegradable polycarbonate.

40. The method according to claim 35, wherein the outer surface of the particle is provided with functional groups.

41. The method according to claim 40, wherein said functional groups are amine groups, carboxyl groups, hydroxyl groups, sulfate groups or amino acids.
